(19) United States
(12) Patent Application Publication
(10) Pub. No.: US 2025/0278022 A1
(43) Pub. Date: Sep. 4, 2025

(54) COMPOUND, POLYMERIC COMPOUND, RESIST COMPOSITION, AND METHOD OF FORMING RESIST PATTERN

(71) Applicant: SAMSUNG ELECTRONICS CO., LTD., Suwon-si (KR)

(72) Inventor: Issei Suzuki, Yokohama (JP)

(21) Appl. No.: 19/008,294

(22) Filed: Jan. 2, 2025

(30) Foreign Application Priority Data
Mar. 1, 2024 (JP) 2024-031133

(51) Int. Cl. G03F 7/039; C07D 221/14; C08F 220/38; G03F 7/004; G03F 7/038

(52) U.S. Cl. CPC G03F 7/0397; C07D 221/14; C08F 220/385; G03F 7/0046; G03F 7/0388

(57) ABSTRACT

Provided are compounds represented by General Formula 1, polymeric compounds having a structural unit derived from the compounds, resist compositions including the polymeric compounds, and method of forming resist patterns by using the resist compositions.

General Formula 1 is the following:

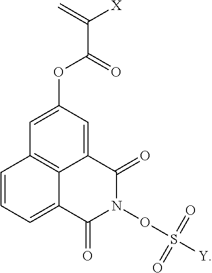

In General Formula 1, X is a hydrogen atom or a methyl group. Y is an alkyl group, a cycloalkyl group, a cycloalkylalkyl, or an aryl group.

### CROSS-REFERENCE TO RELATED APPLICATION

This application claims priority under 35 U.S.C. § 119 to Japanese Patent Application No. 2024-031133, filed on Mar. 1, 2024, in the Japanese Intellectual Property Office, the entirety of which is incorporated by reference herein.

### BACKGROUND

Recently, advances in lithography technology for fabricating semiconductor devices or liquid-crystal display devices have led to rapid pattern miniaturization. To form finer-sized patterns, a reduction in wavelengths (an increase in energy) of exposure light sources has been generally performed.

Resist materials require lithographic characteristics such as sensitivity to exposure light sources, resolution to reproduce fine-sized patterns, and the like. According to the related art, chemically amplified resist compositions, which include components having a change in solubility thereof in developers due to the action of acids and acid generator components generating acids due to exposure to light, have been used as resist materials satisfying such requirements.

In the case of chemically amplified resist compositions, resins having a plurality of structural units are generally used to improve the lithographic characteristics and the like.

### SUMMARY

For purposes of this disclosure, it has been recognized that, when resist patterns are formed, the behavior of acids generated by acid generator components due to exposure to light is a factor significantly affecting the lithographic characteristics. Aspects of this disclosure provide compounds, polymeric compounds, resist compositions, and method of forming resist patterns, which are capable of improving sensitivity and resolution.

According to some aspects of the present disclosure, there is provided a compound represented by General Formula 1,

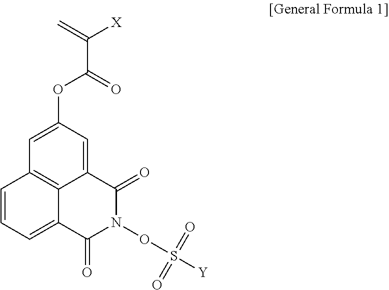

wherein, in General Formula 1,
X is a hydrogen atom or a methyl group, and
Y is a C1 to C20 linear alkyl group in which at least one hydrogen atom is substituted with a fluorine atom, a C3 to C20 branched alkyl group in which at least one hydrogen atom is substituted with a fluorine atom, an unsubstituted C3 to C20 cycloalkyl group, a C3 to C20 cycloalkyl group in which at least one hydrogen atom is substituted with a fluorine atom, a C3 to C20 cycloalkyl group in which at least one hydrogen atom is substituted with an oxygen atom, an unsubstituted C4 to C21 cycloalkylalkyl group, a C4 to C21 cycloalkylalkyl group in which at least one hydrogen atom is substituted with an oxygen atom, or a C6 to C18 aryl group in which at least one hydrogen atom is substituted with a fluorine atom.

According to some aspects of the present disclosure, there is provided a polymeric compound including a first structural unit derived from the compound represented by General Formula 1.

According to some aspects of the present disclosure, there is provided a resist composition including the polymeric compound and an organic solvent.

According to some aspects of the present disclosure, there is provided a method of forming a resist pattern, the method including forming a resist film on a substrate by using the resist composition, exposing the resist film to light, and forming the resist pattern by developing the resist film after the exposing of the resist film to light.

### DETAILED DESCRIPTION

Hereinafter, examples according to the present disclosure will be described in detail. However, the scope of the present disclosure is not limited to the following examples, and various modifications and changes may be made thereto without departing from the scope of the disclosure. Examples described herein may be arbitrarily combined to produce other examples. Herein, unless otherwise stated, operations and measurements of properties and the like are performed at room temperature (about 20° C. to about 25° C.) and relative humidity of about 40% to about 50%.

Unless otherwise stated, the term “alkyl group” used herein includes linear and branched monovalent saturated hydrocarbon groups. The term “cycloalkyl group” used herein includes cyclic monovalent saturated hydrocarbon groups (alicyclic groups). The term “cycloalkylalkyl group” used herein includes groups in which a hydrogen atom of an alkyl group is substituted with a cycloalkyl group. The term “aryl group” used herein includes monovalent aromatic hydrocarbon groups.

As used herein, the term “(meth)acryl” is a collective term including acryl and methacryl and refers to at least one of acryl and methacryl. Likewise, the term “(meth)acrylate” used herein refers to at least one of acrylate and methacrylate.

As used herein, the term “structural unit” refers to a monomer unit constituting a polymeric compound (which may be referred to as a resin, a polymer, or a copolymer).

As used herein, the term “active ray” or the term “radiation” refers to, for example, bright-line spectra of mercury lamps, far-ultraviolet light represented by light of excimer lasers, extreme ultraviolet (EUV) light, X-rays, or electron beams. As used herein, the term “light” refers to active rays or radiation.

Unless otherwise stated, the term “exposure to light” (or the term “light-exposure”) includes lithography by particle beams, such as electron beams or ion beams, as well as exposure to light, such as bright-line spectra of mercury lamps, far-ultraviolet light represented by light of excimer lasers (ArF excimer lasers or the like), EUV light, X-rays, or the like.

Herein, in some structures represented by chemical formulae, there is asymmetric carbon and there may be enantiomers or diastereomers. In such cases, such isomers are expressed by one chemical formula as a representative. These isomers may be used alone or in a mixture thereof.

### [Compound]

A compound according to some implementations of the present disclosure is represented by General Formula 1 shown below.

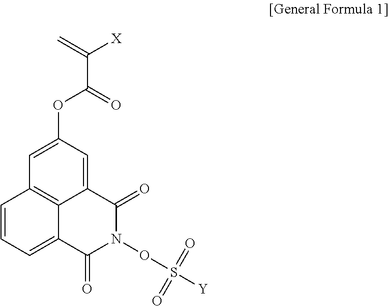

In General Formula 1,
X is a hydrogen atom or a methyl group, and
Y is a C1 to C20 linear alkyl group in which at least one hydrogen atom is substituted with a fluorine atom, a C3 to C20 branched alkyl group in which at least one hydrogen atom is substituted with a fluorine atom, an unsubstituted C3 to C20 cycloalkyl group, a C3 to C20 cycloalkyl group in which at least one hydrogen atom is substituted with a fluorine atom, a C3 to C20 cycloalkyl group in which at least one hydrogen atom is substituted with an oxygen atom, an unsubstituted C4 to C21 cycloalkylalkyl group, a C4 to C21 cycloalkylalkyl group in which at least one hydrogen atom is substituted with an oxygen atom, or a C6 to C18 aryl group in which at least one hydrogen atom is substituted with a fluorine atom.

Compounds having the configuration described above may improve the sensitivity and resolution of a resist composition.

A mechanism, by which the effects set forth above may be achieved by the compound, is as follows. However, the following mechanism is only an example and should not be understood as absolutely limiting the present disclosure.

In resist compositions including photoacid generators, according to the related art, because a phenomenon of partial aggregation of photoacid generators occurs, photosensitive sites (photosensitive points) tend to decrease and become non-uniform. On the other hand, the compound represented by General Formula 1 has a photoacid generating group in a molecule thereof, and the photoacid generating group, which is a photosensitive site, is almost uniformly dispersed in a polymeric compound obtained by the polymerization of the compound represented by General Formula 1. Therefore, photosensitive sites in the polymeric compound are increased, and the rate of change in the solubility of the polymeric compound, which is included in a light-exposed area, in a developer increases, thereby improving the sensitivity thereof.

In addition, in resist compositions (in particular, positive resist compositions) including photoacid generators, according to the related art, because the photoacid generators in a non-light-exposed area have low molecular weights, the dissolution rate of the non-light-exposed area in a developer increases, and thus, it is difficult to make a difference in dissolution rate between the non-light-exposed area and the light-exposed area. On the other hand, use of polymeric compounds obtained by the polymerization of the compound represented by General Formula 1 allows reduction of an amount of low-molecular-weight components in the resist composition, which may reduce the dissolution rate of the non-light-exposed area. Therefore, the difference in dissolution rate between the light-exposed area and the non-light-exposed area in a developer is increased (that is, the dissolution contrast is increased), thereby improving the resolution of the resist composition.

In General Formula 1, examples of the C1 to C20 linear alkyl group, which may constitute Y, and in which at least one hydrogen atom is substituted with a fluorine atom, and the C3 to C20 branched alkyl group, which may constitute Y, and in which at least one hydrogen atom is substituted with a fluorine atom, may include the following groups. For example, examples of the C1 to C20 linear alkyl group and the C3 to C20 branched alkyl group may include a trifluoromethyl group (a perfluoromethyl group), a difluoromethyl group, a pentafluoroethyl group (a perfluoroethyl group), a 2,2,2-trifluoroethyl group, a 1,1,2,2-tetrafluoropropyl group, an n-heptafluoropropyl group (a perfluoropropyl group), a 2H-perfluoropropyl group, a 3,3,3-trifluoropropyl group, a hexafluoroisopropyl group, a heptafluoroisopropyl group (a perfluoroisopropyl group), a nonafluoroisobutyl group (a perfluoroisobutyl group), an n-nonafluorobutyl group (an n-perfluorobutyl group), a 4H-n-perfluorobutyl group, a 2H-perfluoroisobutyl group, a tert-nonafluorobutyl group, an n-perfluoropentyl group, a 1H,1H,2H,2H-perfluoropentyl group, a perfluoroisopentyl group, an n-perfluorohexyl group, a 1H,1H,2H,2H-perfluorohexyl group, or the like.

In General Formula 1, the unsubstituted C3 to C20 cycloalkyl group, which may constitute Y, may be monocyclic or polycyclic. The unsubstituted C3 to C20 cycloalkyl group, which may constitute Y, may include, for example, a cyclopropyl group, a cyclobutyl group, a cyclopentyl group, a cyclohexyl group, a cycloheptyl group, a cyclooctyl group, a cyclononyl group, a cyclodecyl group, a bicyclo[1.1.0]butyl group, a bicyclo[1.1.1]pentyl group, a bicyclo[2.1.0]pentyl group, a bicyclo[3.1.0]hexyl group, a bicyclo[2.1.1]hexyl group, a bicyclo[2.2.0]hexyl group, a bicyclo[2.2.1]heptyl (norbornyl) group, a bicyclo[3.1.1]heptyl group, a bicyclo[3.2.0]heptyl group, a bicyclo[4.1.0]heptyl group, a bicyclo[2.2.2]octyl group, a bicyclo[3.2.1]octyl group, a bicyclo[3.3.0]octyl group, a bicyclo[4.1.1]octyl group, a bicyclo[4.2.0]octyl group, a bicyclo[5.1.0]octyl group, a bicyclo[3.2.2]nonyl group, a bicyclo[3.3.1]nonyl group, a bicyclo[4.2.1]nonyl group, a bicyclo[4.3.0]nonyl group, a bicyclo[5.1.1]nonyl group, a bicyclo[5.2.0]nonyl group, a bicyclo[6.1.0]nonyl group, a bicyclo[4.3.1]decyl group, a tricyclo[5.2.1.0^{2,6}]decyl group, an isobornyl group, an adamantyl group, an androstanyl group, or the like.

In General Formula 1, the C3 to C20 cycloalkyl group, which may constitute Y, and in which at least one hydrogen atom is substituted with a fluorine atom, may be monocyclic or polycyclic. The C3 to C20 cycloalkyl group, in which at least one hydrogen atom is substituted with a fluorine atom, may include, for example, a 1-fluorocyclopropyl group, a 2-fluorocyclopropyl group, a 2,2-difluorocyclopropyl group, a 2,3-difluorocyclopropyl group, a 2,2-difluorocyclobutyl group, a 3,3-difluorocyclobutyl group, a 2,2-difluorocyclopentyl group, a 3,3-difluorocyclopentyl group, a 3,3-difluorocyclohexyl group, a 4,4-difluorocyclohexyl group, a 4,4-difluorocycloheptyl group, a 4,4-difluorocyclooctyl group, or the like.

In General Formula 1, the C3 to C20 cycloalkyl group, which may constitute Y, and in which at least one hydrogen atom is substituted with an oxygen atom, may be monocyclic or polycyclic. The C3 to C20 cycloalkyl group, in which at least one hydrogen atom is substituted with an oxygen atom, may include, for example, a 1,4-epoxycyclohexyl group (a 7-oxabicyclo[2.2.1]heptan-2-yl group), a 3,4-epoxycyclohexyl group, or the like.

In General Formula 1, a cycloalkyl group of the unsubstituted C4 to C21 cycloalkylalkyl group that may constitute Y may be monocyclic or polycyclic. The cycloalkyl group of the unsubstituted C4 to C21 cycloalkylalkyl group may include, for example, a cyclopropylmethyl group, a 2-cyclopropylethyl group, a 1-cyclopropylethyl group, a 3-cyclopropylpropyl group, a 4-cyclopropylbutyl group, a 5-cyclopropylpentyl group, a 6-cyclopropylhexyl group, a 2-cyclopropyl-1-methylethyl group, a cyclobutylmethyl group, a cyclopentylmethyl group, a cyclohexylmethyl group, a cycloheptylmethyl group, a cyclooctylmethyl group, a cyclononylmethyl group, a cyclodecylmethyl group, a norbornan-1-ylmethyl group, a norbornan-2-ylmethyl group, an adamantan-1-ylmethyl group, an adamantan-2-ylmethyl group, or the like.

In General Formula 1, a cycloalkyl group of the C4 to C21 cycloalkylalkyl group, which may constitute Y, and in which at least one hydrogen atom is substituted with an oxygen atom, may be monocyclic or polycyclic. The cycloalkyl group of the C4 to C21 cycloalkylalkyl group, in which at least one hydrogen atom is substituted with an oxygen atom, may include, for example, a 1,4-epoxycyclohexylmethyl group (a 7-oxabicyclo[2.2.1]heptan-2-ylmethyl group), a 3,4-epoxycyclohexylmethyl group, a 3,4-epoxycyclohexylethyl group, a camphanyl group, or the like.

In General Formula 1, the C6 to C18 aryl group, which may constitute Y, and in which at least one hydrogen atom is substituted with a fluorine atom, may be monocyclic or polycyclic. The C6 to C18 aryl group, in which at least one hydrogen atom is substituted with a fluorine atom, may include, for example, a 2-fluorophenyl group, a 3-fluorophenyl group, a 4-fluorophenyl group, a 2,4-difluorophenyl group, a 2,3-difluorophenyl group, a 3,4-difluorophenyl group, a 3,5-difluorophenyl group, a 2,4,5-trifluorophenyl group, a 2,3,4-trifluorophenyl group, a 2,3,4,5-tetrafluorophenyl group, a 2,3,5,6-tetrafluorophenyl group, a 2,3,4,5,6-pentafluorophenyl group, a 2-fluoronaphthyl group, a 3-fluoronaphthyl group, a 4-fluoronaphthyl group, a 2,3,4,5,6,7-hexafluoronaphthyl group, a 2,3,4,5,6,7,8-heptafluoronaphthyl group, a 1,3,4,5,6,7,8-heptafluoronaphthyl group, a 4,4′-difluorobiphenyl group, or the like.

In some implementations, in General Formula 1, Y is a C1 to C20 linear alkyl group in which at least one hydrogen atom is substituted with a fluorine atom, a C3 to C20 branched alkyl group in which at least one hydrogen atom is substituted with a fluorine atom, an unsubstituted C3 to C20 cycloalkyl group, a C3 to C20 cycloalkyl group in which at least one hydrogen atom is substituted with a fluorine atom, or a C6 to C18 aryl group in which at least one hydrogen atom is substituted with a fluorine atom. In General Formula 1, when Y has a structure substituted with a fluorine atom, because the strength of a generated acid is relatively great, the polymeric compound has a relatively high rate of change of the solubility thereof in a developer, thereby improving the sensitivity thereof and improving the solubility thereof in an organic solvent. In addition, in General Formula 1, when Y includes a cyclic group, because the volume of Y is great in a stereoscopic viewpoint, the diffusivity of a generated acid is suppressed, thereby improving the resolution of the resist composition. Considering the points described above, Y in General Formula 1 may be a C1 to C20 linear alkyl group in which at least one hydrogen atom is substituted with a fluorine atom, a C3 to C20 branched alkyl group in which at least one hydrogen atom is substituted with a fluorine atom, a C3 to C20 cycloalkyl group in which at least one hydrogen atom is substituted with a fluorine atom, or a C6 to C18 aryl group in which at least one hydrogen atom is substituted with a fluorine atom.

Examples of the compound represented by General Formula 1 include Compounds 1 to 26 shown below.

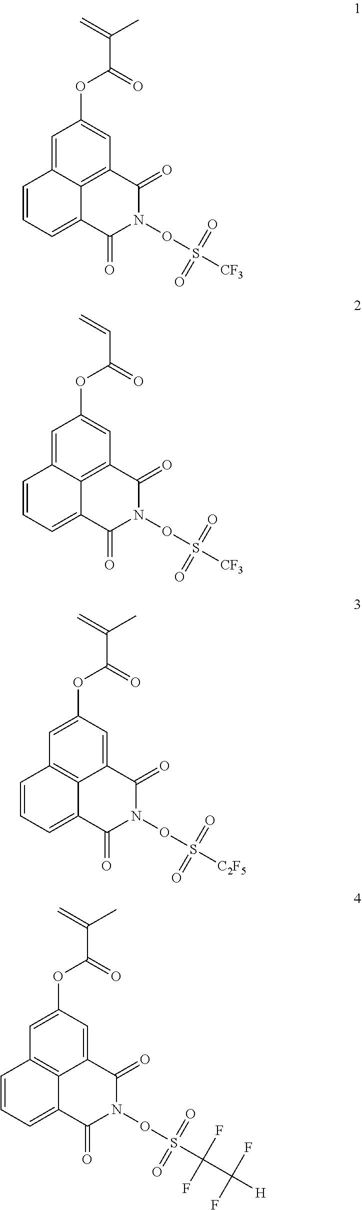

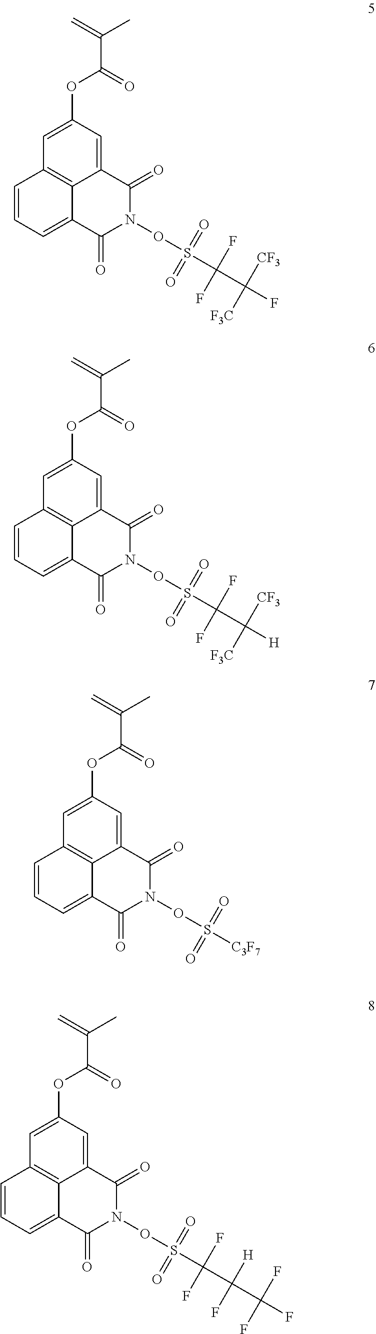

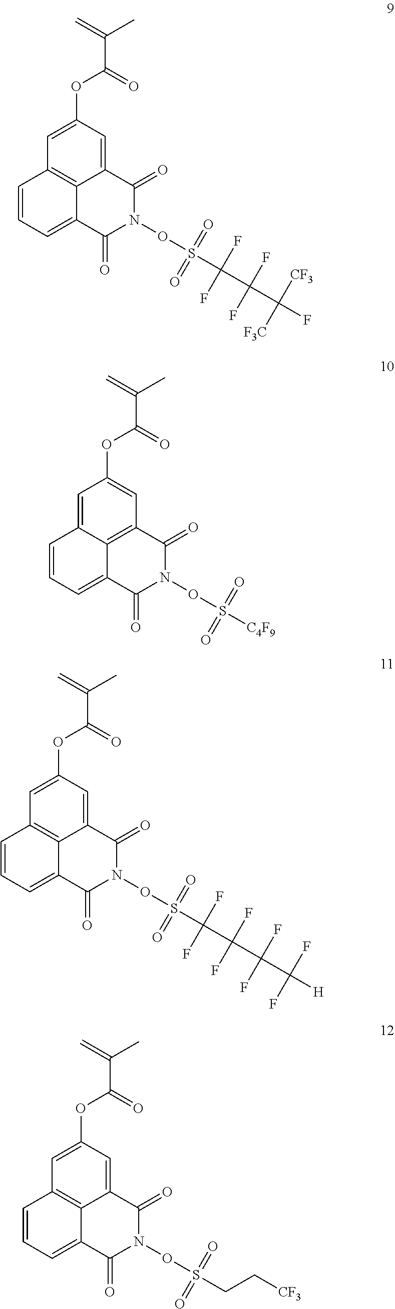

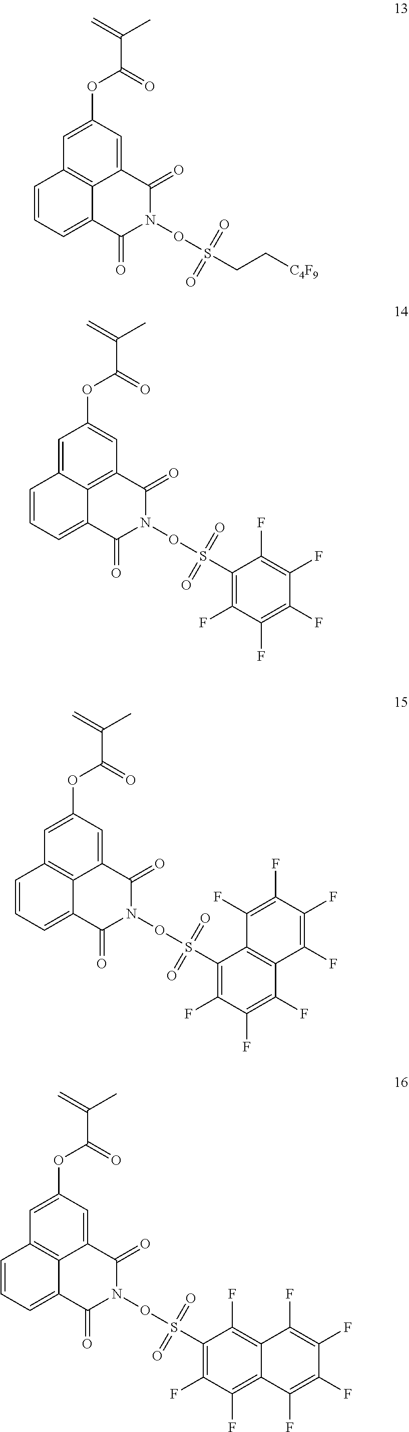

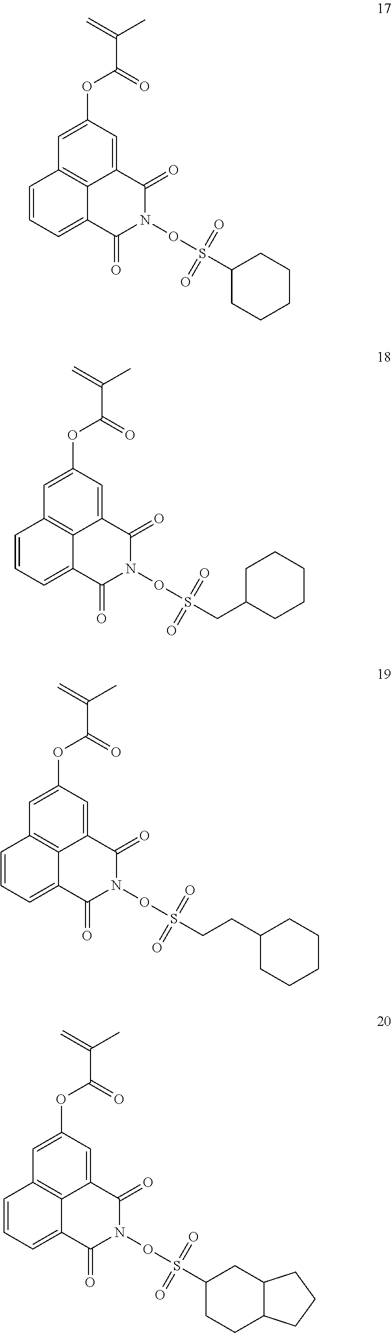

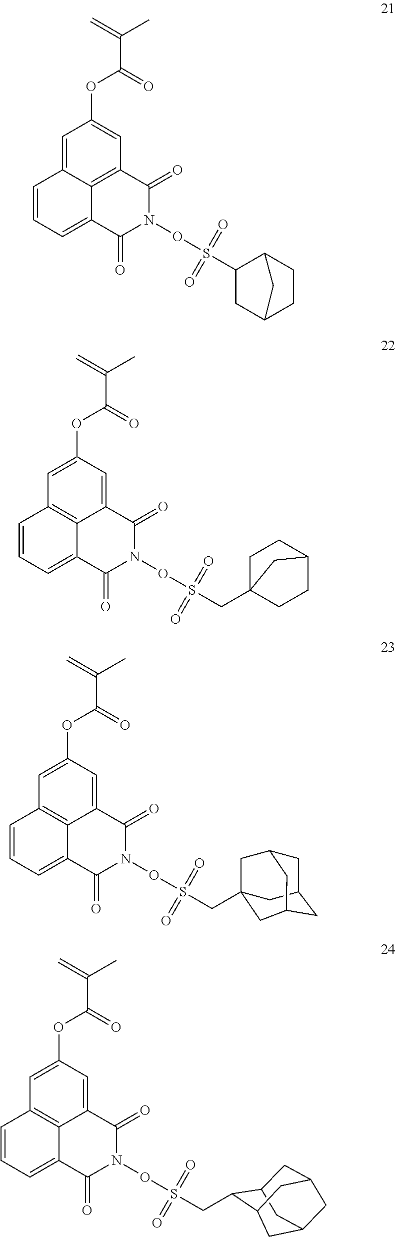

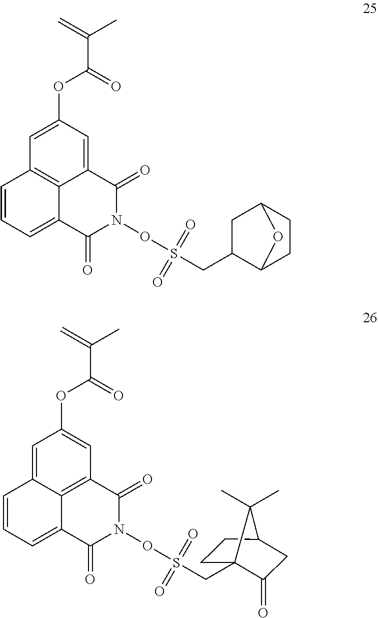

In some implementations, Compound 1, Compound 2, Compound 10, Compound 14, Compound 21, or Compound 26, from among the compounds set forth above, may be used. For example, Compound 1, Compound 2, Compound 10, or Compound 14, from among the compounds set forth above, may be used. The synthesis of these examples of compounds is described below.

A synthesis method of the compound represented by General Formula 1 is not particularly limited. In some implementations, the compound represented by General Formula 1 is synthesized by a method in which a sulfonic acid ester derivative of an N-hydroxyimide compound is obtained by reacting N-hydroxynaphthalimide with sulfonyl halide, and then, a (meth)acryloyloxy group is introduced into a naphthalene ring. More specific synthesis methods of the compound represented by General Formula 1 may be easily understood by those of ordinary skill in the art with reference to the examples described below.

### [Polymeric Compound]

According to some implementations of the present disclosure, there is provided a polymeric compound (A) (which may be simply referred to as a component (A), hereinafter) including a structural unit that is derived from the compound represented by General Formula 1. The structural unit (which may be simply referred to as a structural unit (O) or a first structural unit, hereinafter) derived from the compound represented by General Formula 1 may have a photoacid generating group and thus may improve the sensitivity and resolution of a resist composition. The structural unit (O) in the component (A) may be used alone or in a combination of two or more structural units (O).

The structural unit (O) may be a bivalent structural unit generated by the cleavage of an ethylenically unsaturated double bond in the compound represented by General Formula 1. In some implementations of the structural unit (O), because a structure except for the structure obtained by the cleavage of the ethylenically unsaturated double bond is the same as that in General Formula 1, descriptions thereof are omitted.

The component (A) may have one or more other structural units in addition to the structural unit (O). Hereinafter, an example of another such structural unit is described.

### Structural Unit Having Acid-Decomposable Group

The component (A) may have a structural unit (which may be simply referred to as a structural unit (L) or a second structural unit, hereinafter) including an acid-decomposable group that has increasing polarity due to the action of an acid. The term “acid-decomposable group” refers to a group having acid-decomposability that allows at least some bonds in the structure of the acid-decomposable group to be cleaved.

The acid-decomposable group, which has increasing polarity due to the action of an acid, may include, for example, a group dissociated by the action of an acid and generating a polar group. The polar group may include, for example, a carboxyl group, a hydroxy group, an amino group, a sulfonic acid group (—SO₃H), or the like.

For example, the acid-decomposable group may include a group in which the polar group is protected by an acid-labile group (for example, a group in which a hydrogen atom of an OH-containing polar group is protected by an acid-labile group).

The term “acid-labile group” refers to both a group having acid-lability that allows a bond between the acid-labile group and an atom adjacent to the acid-labile group to be cleaved due to the action of an acid, and a group in which some bonds of the acid-labile group are cleaved due to the action of an acid and then a decarboxylation reaction additionally occurs, whereby a bond between the acid-labile group and an atom adjacent to the acid-labile group may be cleaved.

The acid-labile group constituting the acid-decomposable group may be a group having polarity that is lower than that of a polar group generated due to the dissociation of the acid-labile group. By such a configuration, when the acid-labile group is dissociated due to the action of an acid, a polar group having higher polarity than the acid-labile group is generated, thereby having increased polarity. As a result, the total polarity of the component (A) increases. As the polarity of the component (A) increases, the solubility thereof in a developer relatively changes. When the developer includes an alkaline developer, the solubility of the component (A) increases, and when the developer includes an organic developer, the solubility thereof decreases.

Examples of the acid-labile group include acid-labile groups proposed so far as acid-labile groups of base resins for chemically amplified resist compositions. Examples of the acid-labile groups of the base resins for chemically amplified resist compositions include an “acetal-type acid-labile group”, a “tertiary alkyl ester-type acid-labile group”, and a “tertiary alkyloxycarbonyl acid-labile group”.

### (Acetal-Type Acid-Labile Group)

The acid-labile group, which protects a carboxyl group or a hydroxyl group of the polar group, may include, for example, an acid-labile group (which may be referred to as an “acetal-type acid-labile group”, hereinafter) represented by Formula (L-r-1) shown below.

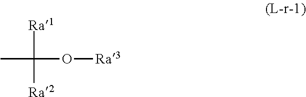

In Formula (L-r-1), Ra′¹ and Ra′² are each independently a hydrogen atom or an alkyl group and Ra′³ is a hydrocarbon group, wherein Ra′³ may be bonded to one of Ra′¹ and Ra′² to form a ring.

In some implementations, in Formula (L-r-1), at least one of Ra′¹ and Ra′² may be a hydrogen atom. For example, in Formula (L-r-1), both Ra′¹ and Ra′² may be hydrogen atoms.

When Ra′¹ or Ra′² is an alkyl group, the alkyl group may include a C1 to C5 alkyl group. The alkyl group may include a linear or branched alkyl group, for example, a methyl group, an ethyl group, a propyl group, an isopropyl group, an n-butyl group, an isobutyl group, a tert-butyl group, a pentyl group, an isopentyl group, a neopentyl group, or the like.

In Formula (L-r-1), the hydrocarbon group for Ra′³ may include a linear or branched alkyl group or a cyclic hydrocarbon group.

The linear alkyl group may include a C1 to C5 linear alkyl group, for example, a methyl group, an ethyl group, an n-propyl group, an n-butyl group, an n-pentyl group, or the like.

The branched alkyl group may include a C3 to C10 branched alkyl group, for example, an isopropyl group, an isobutyl group, a tert-butyl group, an isopentyl group, a neopentyl group, a 1,1-diethylpropyl group, a 2,2-dimethylbutyl group, or the like.

When Ra′³ is a cyclic hydrocarbon group, the hydrocarbon group may include an aliphatic hydrocarbon group, an aromatic hydrocarbon group, a polycyclic group, or a monocyclic group.

The aliphatic hydrocarbon group that is monocyclic may include a group obtained by removing one hydrogen atom from a monocycloalkane. The monocycloalkane may include a C3 to C6 monocycloalkane, for example, cyclopentane, cyclohexane, or the like.

The aliphatic hydrocarbon group that is polycyclic may include a group obtained by removing one hydrogen atom from a polycycloalkane. The polycycloalkane may include a C7 to C12 polycycloalkane, for example, adamantane, norbornane, isobornane, tricyclodecane, tetracyclododecane, or the like.

When the cyclic hydrocarbon group for Ra′³ includes an aromatic hydrocarbon group, the aromatic hydrocarbon group includes a hydrocarbon group having at least one aromatic ring.

The aromatic ring is not particularly limited as long as the aromatic ring is a cyclic conjugated system having 4n+2 π-electrons, and the aromatic ring may be monocyclic or polycyclic. The aromatic ring may have 5 to 30 carbon atoms. For example, the aromatic ring may include: an aromatic hydrocarbon ring, such as benzene, naphthalene, anthracene, or phenanthrene; an aromatic heterocycle in which some of carbon atoms constituting an aromatic hydrocarbon ring are substituted with heteroatoms; or the like. The heteroatoms in the aromatic heterocycle may include oxygen atoms, sulfur atoms, nitrogen atoms, or the like. For example, the aromatic heterocycle may include a pyridine ring, a thiophene ring, or the like.

For example, the aromatic hydrocarbon group for Ra′³ may include: a group (an aryl group or a heteroaryl group) obtained by removing one hydrogen atom from an aromatic hydrocarbon ring or an aromatic heterocycle; a group obtained by removing one hydrogen atom from an aromatic compound (for example, biphenyl, fluorene, or the like) including two or more aromatic rings; a group (for example, arylalkyl group, such as a benzyl group, a phenethyl group, a 1-naphthylmethyl group, a 2-naphthylmethyl group, a 1-naphthylethyl group, or a 2-naphthylethyl group, or the like) in which one of hydrogen atoms of an aromatic hydrocarbon ring or an aromatic heterocycle is substituted with an alkylene group; or the like. The alkylene group bonded to the aromatic hydrocarbon ring or the aromatic heterocycle may have 1 to 4 carbon atoms.

The cyclic hydrocarbon group for Ra′³ may have a substituent. The substituent may include, for example, —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN, —R^{P2}—COOH (hereinafter, these substituents may be collectively referred to as “Ra^{x5}”), or the like.

Here, R^{P1} is a C1 to C10 acyclic saturated hydrocarbon group, a C3 to C20 aliphatic cyclic saturated hydrocarbon group, or a C6 to C30 aromatic hydrocarbon group. In addition, R^{P2} is a single bond, a C1 to C10 acyclic saturated hydrocarbon group, a C3 to C20 aliphatic cyclic saturated hydrocarbon group, or a C6 to C30 aromatic hydrocarbon group. Here, some or all of hydrogen atoms of each of the acyclic saturated hydrocarbon group, the aliphatic cyclic saturated hydrocarbon group, and the aromatic hydrocarbon group for R^{P1} and R^{P2} may be substituted with fluorine atoms. The aliphatic cyclic saturated hydrocarbon group may include one or more substituents of a single type or one or more substituents of each of a plurality of types.

When Ra′³ is bonded to one of Ra′¹ and Ra′² to form a ring, the cyclic group that is formed may have a 4-membered ring, a 5-membered ring, a 6-membered ring, or a 7-membered ring. The cyclic group may include, for example, a tetrahydropyranyl group, a tetrahydrofuranyl group, or the like.

### (Tertiary Alkyl Ester-Type Acid-Labile Group)

The acid-labile group, which protects the carboxyl group of the polar group, may include, for example, an acid-labile group represented by Formula (L-r-2).

An acid-labile group including an alkyl group, among acid-labile groups represented by Formula (L-r-2), is also referred to as a “tertiary alkyl ester-type acid-labile group” for convenience, hereinafter.

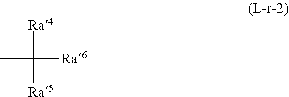

In Formula (L-r-2), Ra′⁴, Ra′⁵, and Ra′⁶ are each independently a hydrocarbon group, and Ra′⁵ and Ra′⁶ may be bonded to each other to form a ring.

The hydrocarbon group for Ra′⁴ may include a linear or branched alkyl group, an acyclic or cyclic alkenyl group, or a cyclic hydrocarbon group.

Examples of the linear or branched alkyl group and the cyclic alkenyl group (a monocyclic aliphatic hydrocarbon group, a polycyclic aliphatic hydrocarbon group, or an aromatic hydrocarbon group) may be the same as those disclosed for Ra′³.

The acyclic or cyclic alkenyl group for Ra′⁴ may include a C2 to C10 alkenyl group.

Examples of the hydrocarbon group for each of Ra′⁵ and Ra′⁶ may be the same as those disclosed for Ra′³.

When Ra′⁵ and Ra′⁶ are bonded to each other to form a ring, examples of the tertiary alkyl ester-type acid-labile group may include a group represented by Formula (L-r2-1), a group represented by Formula (L-r2-2), and a group represented by Formula (L-r2-3).

When Ra′⁴, Ra′⁵, and Ra′⁶ are not bonded to each other and are each independently a hydrocarbon group, the tertiary alkyl ester-type acid-labile group may include, for example, a group represented by Formula (L-r2-4).

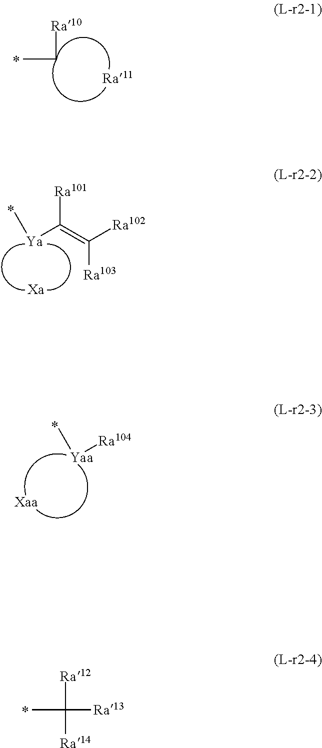

In Formula (L-r2-1), Formula (L-r2-2), Formula (L-r2-3), and Formula (L-r2-4), “*” represents a binding site.

In Formula (L-r2-1), Ra′¹⁰ represents a C1 to C12 linear or branched alkyl group in which at least a portion may be substituted with a halogen atom or a heteroatom-containing group. Ra′¹¹ represents a group that forms, together with a carbon atom bonded to Ra′¹⁰, an aliphatic cyclic group. In Formula (L-r2-2), Ya is a carbon atom. Xa is a group that forms, together with Ya, a cyclic hydrocarbon group. Some or all of hydrogen atoms in the cyclic hydrocarbon group may be substituted. Ra¹⁰¹, Ra¹⁰², and Ra¹⁰³ are each independently a hydrogen atom, a C1 to C10 acyclic saturated hydrocarbon group, or a C3 to C20 alicyclic hydrocarbon group. Some or all of hydrogen atoms in each of the acyclic saturated hydrocarbon group and the alicyclic hydrocarbon group may be substituted. Two or more of Ra¹⁰¹, Ra¹⁰², and Ra¹⁰³ may be bonded to each other to form a cyclic structure. In Formula (L-r2-3), Yaa may be a carbon atom. Xaa is a group that forms, together with Yaa, an alicyclic group. Ra¹⁰⁴ is an aromatic hydrocarbon group that may have a substituent. In Formula (L-r2-4), Ra′¹² and Ra′¹³ are each independently a C1 to C10 acyclic saturated hydrocarbon group. Some or all of hydrogen atoms in the acyclic saturated hydrocarbon group may be substituted. Ra′¹⁴ is a hydrocarbon group that may have a substituent.

In Formula (L-r2-1), Ra′¹⁰ is a C1 to C12 linear or branched alkyl group in which a portion thereof may be substituted with a halogen atom or a heteroatom-containing group.

The linear alkyl group for Ra′¹⁰ has 1 to 12 carbon atoms. Examples of the branched alkyl group for Ra′¹⁰ may be the same as those for Ra′³.

In the alkyl group for Ra′¹⁰, a portion thereof may be substituted with a halogen atom or a heteroatom-containing group. For example, some of hydrogen atoms constituting the alkyl group may be substituted with halogen atoms or heteroatom-containing groups. In addition, some of carbon atoms (a methylene group or the like) constituting the alkyl group may be substituted with heteroatom-containing groups.

The heteroatom set forth above may include an oxygen atom, a sulfur atom, a nitrogen atom. The heteroatom-containing group may include O—, —C(═O)—O—, —O—C(═O)—, —C(═O)—, —O—C(═O)—O—, —C(═O)—NH—, —NH—, —S—, —S(═O)₂—, —S(═O)₂—O—, or the like.

In some implementations, in Formula (L-r2-1), Ra′¹¹ (which is a group forming, together with a carbon atom bonded to Ra′¹⁰, an alicyclic group) may be a group listed as an example of a monocyclic or polycyclic aliphatic hydrocarbon group (an alicyclic hydrocarbon group) for Ra′³ in Formula (L-r-1). For example, Ra′¹¹ may be a monocyclic alicyclic hydrocarbon group. For example, Ra′¹¹ may be a cyclopentyl group or a cyclohexyl group.

In Formula (L-r2-2), the cyclic hydrocarbon group, which is formed by Xa and Ya together, may include a group obtained by further removing one or more hydrogen atoms from a monovalent alicyclic hydrocarbon group for Ra′³ in Formula (L-r-1).

The cyclic hydrocarbon group, which is formed by Xa and Ya together, may have a substituent. Examples of the substituent may be the same as those of the substituent that may be included in the cyclic hydrocarbon group for Ra′³.

In Formula (L-r2-2), a C1 to C10 monovalent acyclic saturated hydrocarbon group for each of Ra¹⁰¹, Ra¹⁰², and Ra¹⁰³ may include, for example, a methyl group, an ethyl group, a propyl group, a butyl group, a pentyl group, a hexyl group, a heptyl group, an octyl group, a decyl group, or the like.

A C3 to C20 aliphatic cyclic saturated hydrocarbon group for each of Ra¹⁰¹, Ra¹⁰², and Ra¹⁰³ may include, for example, a monocyclic aliphatic saturated hydrocarbon group, such as a cyclopropyl group, a cyclobutyl group, a cyclopentyl group, a cyclohexyl group, a cycloheptyl group, a cyclooctyl group, a cyclodecyl group, or a cyclododecyl group; a polycyclic alicyclic hydrocarbon group, such as a bicyclo[2.2.2]octanyl group, tricyclo[5.2.1.02,6]decanyl group, a tricyclo[3.3.1.13,7]decanyl group, a tetracyclo[6.2.1.13,6.02,7]dodecanyl group, or an adamantly group; or the like. For example, Ra¹⁰¹, Ra¹⁰², and Ra¹⁰³ may each be a hydrogen atom or a C1 to C10 acyclic saturated hydrocarbon group, in terms of ease of synthesis.

A substituent in the acyclic saturated hydrocarbon group or the alicyclic hydrocarbon group, which is indicated by each of Ra¹⁰¹, Ra¹⁰², and Ra¹⁰³, may include, for example, a same group as listed as the example of Ra^{x5}.

A group including a carbon-carbon double bond, which is generated due to the cyclic structure formed by bonding between two or more of Ra¹⁰¹, Ra¹⁰², and Ra¹⁰³, may include, for example, a cyclopentenyl group, a cyclohexenyl group, a methylcyclopentenyl group, a methylcyclohexenyl group, a cyclopentylideneethenyl group, a cyclohexylideneethenyl group, or the like. The cyclopentenyl group, the cyclohexenyl group, or the cyclopentylideneethenyl group, from among the examples set forth above, may be used in terms of ease of synthesis.

In Formula (L-r2-3), the alicyclic group, which is formed by Xaa and Yaa together, may include a group listed as an example of the monocyclic or polycyclic aliphatic hydrocarbon group for Ra′³ in Formula (L-r-1).

In Formula (L-r2-3), the aromatic hydrocarbon group for Ra¹⁰⁴ may include a group obtained by removing one or more hydrogen atoms from a C5 to C30 aromatic hydrocarbon ring.

In some implementations, Ra¹⁰⁴ is a group obtained by removing one or more hydrogen atoms from a C6 to C15 aromatic hydrocarbon ring. For example, Ra¹⁰⁴ may be a group obtained by removing one or more hydrogen atoms from benzene, naphthalene, anthracene, or phenanthrene.

A substituent, which may be included in Ra¹⁰⁴ in Formula (L-r2-3), may include a methyl group, an ethyl group, a propyl group, a hydroxyl group, a carboxyl group, a halogen atom, an alkoxy group (a methoxy group, an ethoxy group, a propoxy group, a butoxy group, or the like), an alkyloxycarbonyl group, or the like.

In Formula (L-r2-4), Ra′¹² and Ra′¹³ are each independently a C1 to C10 acyclic saturated hydrocarbon group. The C1 to C10 acyclic saturated hydrocarbon group for each of Ra′¹² and Ra′¹³ may include a same group as listed as an example of the C1 to C10 acyclic saturated hydrocarbon group for each of Ra¹⁰¹, Ra¹⁰², and Ra¹⁰³. Some or all of hydrogen atoms in the acyclic saturated hydrocarbon group may be substituted.

In some implementations, Ra′¹² and Ra′¹³ are each be a hydrogen atom or a C1 to C5 alkyl group. For example, Ra′¹² and Ra′¹³ may each be a methyl group or an ethyl group.

When the acyclic saturated hydrocarbon group indicated by each of Ra′¹² and Ra′¹³ is substituted, the substituent may include, for example, a same group as listed as an example of Ra^{x5}.

In Formula (L-r2-4), Ra′¹⁴ is a hydrocarbon group that may have a substituent. The hydrocarbon group for Ra′¹⁴ may include a linear or branched alkyl group or a cyclic hydrocarbon group.

The linear alkyl group for Ra′¹⁴ may include a C1 to C5 linear alkyl group, for example, a methyl group, an ethyl group, an n-propyl group, an n-butyl group, an n-pentyl group, or the like.

The branched alkyl group for Ra′¹⁴ may include a C3 to C10 branched alkyl group, for example, an isopropyl group, an isobutyl group, a tert-butyl group, an isopentyl group, a neopentyl group, a 1,1-diethylpropyl group, or a 2,2-dimethylbutyl group.

When Ra′¹⁴ is a cyclic hydrocarbon group, the cyclic hydrocarbon group may include an aliphatic hydrocarbon group or an aromatic hydrocarbon group and may be polycyclic or monocyclic.

A monocyclic aliphatic hydrocarbon group may include a group obtained by removing one hydrogen atom from a monocycloalkane. The monocycloalkane may include a C3 to C6 monocycloalkane, for example, cyclopentane, cyclohexane, or the like.

A polycyclic aliphatic hydrocarbon group may include a group obtained by removing one hydrogen atom from a polycycloalkane, and the polycycloalkane may include a C7 to C12 polycycloalkane, for example, adamantane, norbornane, isobornane, tricyclodecane, tetracyclododecane, or the like.

The aromatic hydrocarbon group for Ra′¹⁴ may include a same group as listed as an example of the aromatic hydrocarbon group for Ra¹⁰⁴. In some implementations, Ra′¹⁴ is a group obtained by removing one or more hydrogen atoms from a C6 to C15 aromatic hydrocarbon ring. For example, Ra′¹⁴ may be a group obtained by removing one or more hydrogen atoms from benzene, naphthalene, anthracene, or phenanthrene.

The substituent, which may be included in Ra′¹⁴, may include a same substituent as listed as an example of the substituent that may be included in Ra¹⁰⁴.

When Ra′¹⁴ in Formula (L-r2-4) is a naphthyl group, a position at which Ra′¹⁴ is bonded to a tertiary carbon atom in Formula (L-r2-4) may be one of the 1-position and the 2-position of the naphthyl group.

When Ra′¹⁴ in Formula (L-r2-4) is an anthryl group, a position at which Ra′¹⁴ is bonded with a tertiary carbon atom in Formula (L-r2-4) may be one of the 1-position, the 2-position, and the 9-position of the anthryl group.

Examples of the group represented by Formula (L-r2-1) are as follows.

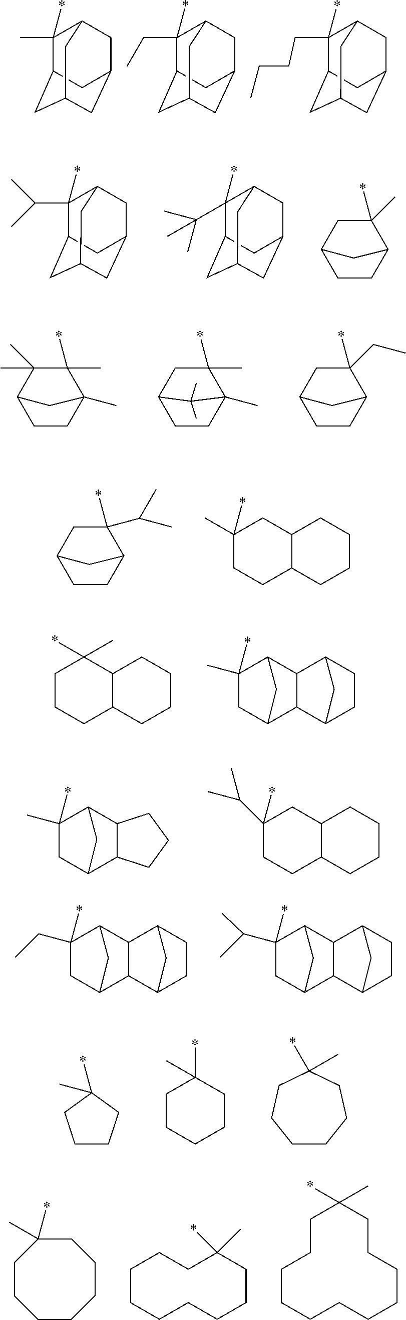

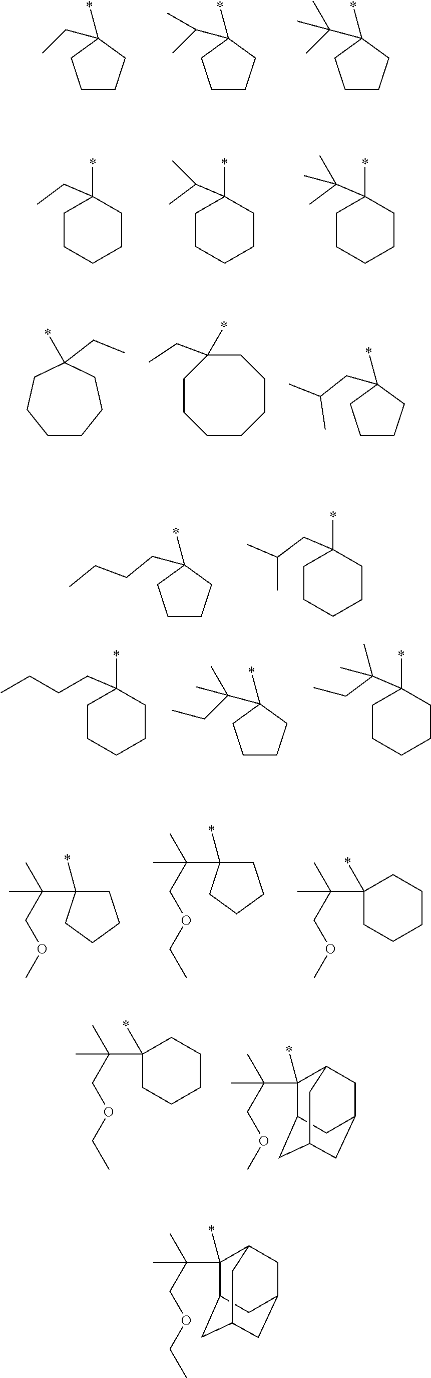

Examples of the group represented by Formula (L-r2-2) are as follows.

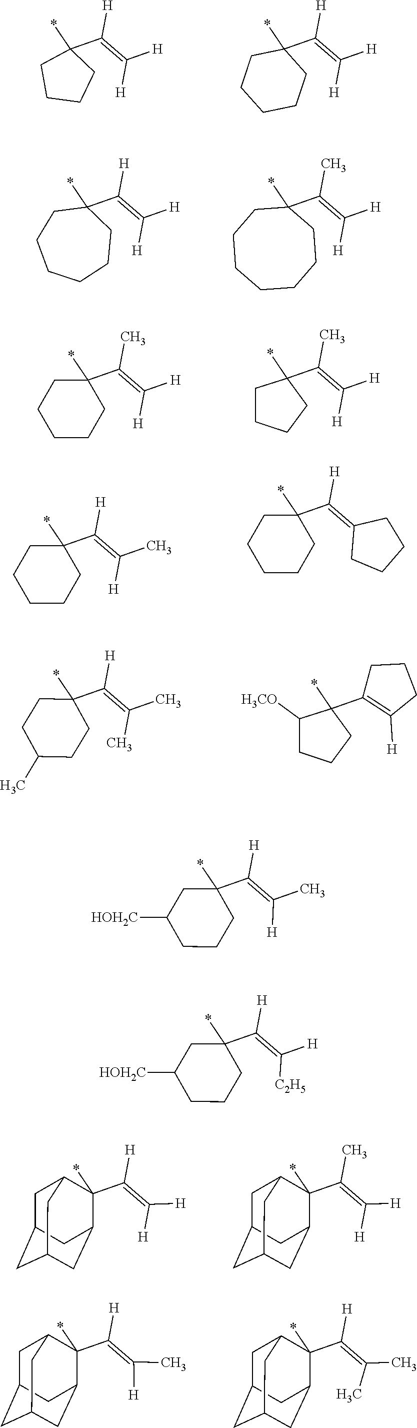

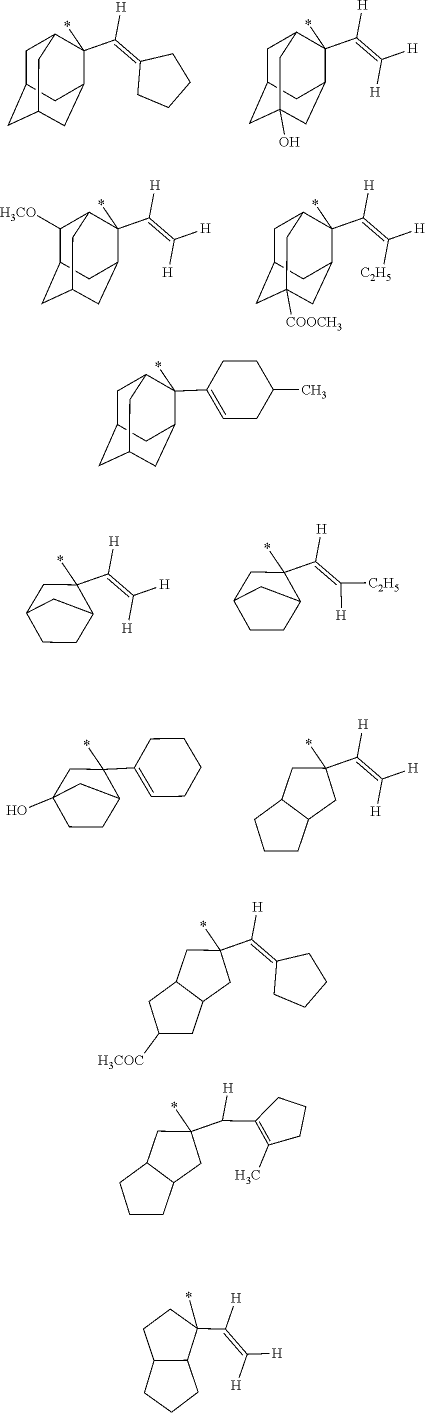

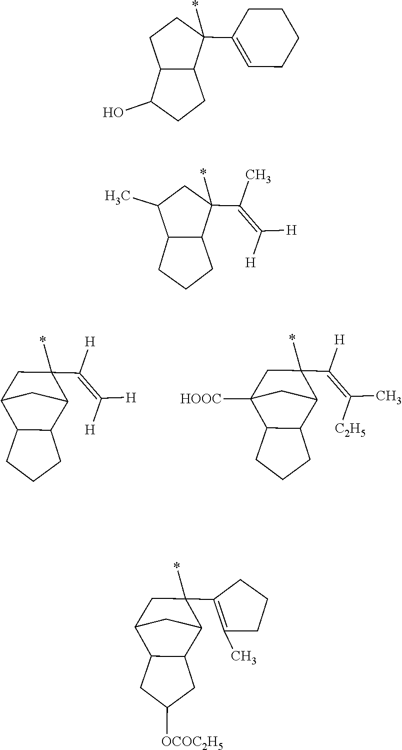

Examples of the group represented by Formula (L-r2-3) are as follows.

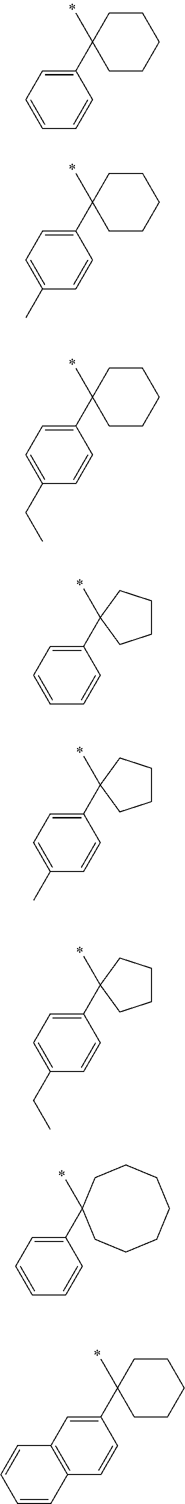

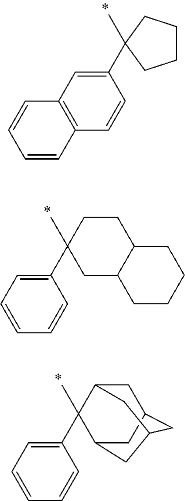

Examples of the group represented by Formula (L-r2-4) are as follows.

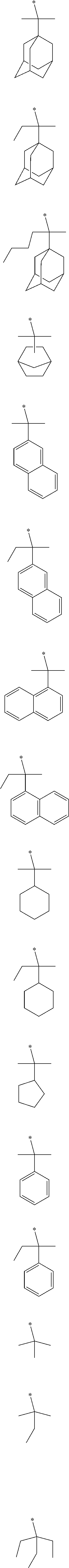

### (Tertiary Alkyloxycarbonyl Acid-Labile Group)

The acid-labile group, which protects the hydroxyl group of the polar group, may include, for example, an acid-labile group (which is also referred to as a “tertiary alkyloxycarbonyl acid-labile group” for convenience, hereinafter) represented by Formula (L-r-3) shown below.

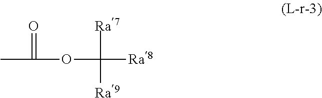

In Formula (L-r-3), Ra′⁷, Ra′⁸, and Ra′⁹ are each independently an alkyl group.

In Formula (L-r-3), Ra′⁷, Ra′⁸, and Ra′⁹ may each be independently a C1 to C5 alkyl group. In addition, the total number of carbon atoms of the respective alkyl groups may be 3 to 7.

The structural unit (L) may include a structural unit derived from an acrylic acid ester in which a hydrogen atom bonded to an α-position carbon atom may be substituted with a substituent, a structural unit derived from acrylamide, a structural unit in which at least some of hydrogen atoms in a hydroxyl group of a structural unit derived from hydroxystyrene or a hydroxystyrene derivative are protected by a substituent including the acid-decomposable group, a structural unit in which at least some of hydrogen atoms in —C(═O)—OH of a structural unit derived from vinylbenzoic acid or a vinylbenzoic acid derivative are protected by a substituent including the acid-decomposable group, or the like.

In some implementations, the structural unit (L) includes, from among the structural units listed above, a structural unit derived from an acrylic acid ester in which a hydrogen atom bonded to an α-position carbon atom may be substituted with a substituent. The structural unit L as such may include, for example, a structural unit represented by Formula (L1-1) or Formula (L1-2) shown below.

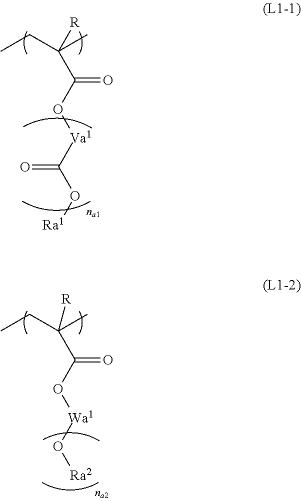

In each of Formula (L1-1) and Formula (L1-2), R is a hydrogen atom, a C1 to C5 alkyl group, or a C1 to C5 alkyl halide group. Va¹ is a hydrocarbon group that may have an ether bond. nₐ₁ is 0, 1, or 2. Ra¹ is an acid-labile group represented by Formula (L-r-1) or Formula (L-r-2). Wa¹ is a (nₐ₂+1)-valent hydrocarbon group, wherein nₐ₂ is 1, 2, or 3 and Ra² is an acid-labile group represented by Formula (L-r-1) or Formula (L-r-3).

In Formula (L1-1), the C1 to C5 alkyl group for R may include a C1 to C5 linear or branched alkyl group, for example, a methyl group, an ethyl group, a propyl group, an isopropyl group, an n-butyl group, an isobutyl group, a tert-butyl group, a pentyl group, an isopentyl group, a neopentyl group, or the like. The C1 to C5 alkyl halide group is a group in which some or all of hydrogen atoms of the C1 to C5 alkyl group are substituted with halogen atoms. The halogen atoms may include fluorine atoms.

In some implementations, R may be a hydrogen atom, a C1 to C5 alkyl group, or a C1 to C5 alkyl fluoride group. For example, R may be a hydrogen atom or a methyl group based on ease of industrial availability.

In Formula (L1-1), the hydrocarbon group for Va¹ may include an aliphatic hydrocarbon group or an aromatic hydrocarbon group.

In some implementations, the aliphatic hydrocarbon group as the hydrocarbon group for Va¹ is saturated or unsaturated. For example, the aliphatic hydrocarbon group as the hydrocarbon group for Va¹ may be saturated.

For example, the aliphatic hydrocarbon group may include a linear aliphatic hydrocarbon group, a branched aliphatic hydrocarbon group, an aliphatic hydrocarbon group including a ring in the structure thereof, or the like. In some implementations, the linear aliphatic hydrocarbon group has 1 to 10 carbon atoms, for example, 1 to 6 carbon atoms.

In some implementations, the linear aliphatic hydrocarbon group includes a linear alkylene group. For example, the linear aliphatic hydrocarbon group may include a methylene group [—CH₂—], an ethylene group [—(CH₂)₂—], a trimethylene group [—(CH₂)₃—], a tetramethylene group [—(CH₂)₄—], a pentamethylene group [—(CH₂)₅—], or the like.

In some implementations, the branched aliphatic hydrocarbon group has 3 to 10 carbon atoms, for example, 3 to 6 carbon atoms.

In some implementations, the branched aliphatic hydrocarbon group includes a branched alkylene group. For example, the branched aliphatic hydrocarbon group may include an alkylalkylene group or the like, the alkylalkylene group including: an alkylmethylene group, such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an alkylethylene group, such as —CH(CH₃)CH₂—, —CH(CH₃)CH(CH₃)—, —C(CH₃)₂CH₂—, —CH(CH₂CH₃)CH₂—, or —C(CH₂CH₃)₂—CH₂—; an alkyltrimethylene group, such as —CH(CH₃)CH₂CH₂— or —CH₂CH(CH₃)CH₂—; an alkyltetramethylene group, such as —CH(CH₃)CH₂CH₂CH₂— or —CH₂CH(CH₃)CH₂CH₂—; or the like. The alkyl group in the alkylalkylene group may include a C1 to C5 linear alkyl group.

The aliphatic hydrocarbon group including a ring in the structure thereof may include an alicyclic hydrocarbon group (a group obtained by removing two hydrogen atoms from an aliphatic hydrocarbon ring), a group in which an alicyclic hydrocarbon group is bonded to an end of a linear or branched aliphatic hydrocarbon group, a group in which an alicyclic hydrocarbon group is interposed in the middle of a linear or branched aliphatic hydrocarbon group, or the like. The linear or branched aliphatic hydrocarbon group may include a same group as listed above as an example of the linear aliphatic hydrocarbon group or the branched aliphatic hydrocarbon group.

In some implementations, the alicyclic hydrocarbon group has 3 to 20 carbon atoms, for example, 3 to 12 carbon atoms. The alicyclic hydrocarbon group may be polycyclic or monocyclic. In some implementations, a monocyclic alicyclic hydrocarbon group is a group obtained by removing two hydrogen atoms from a monocycloalkane. The monocycloalkane may include a C3 to C6 monocycloalkane. For example, the monocycloalkane may include cyclopentane, cyclohexane, or the like. A polycyclic alicyclic hydrocarbon group may be a group obtained by removing two hydrogen atoms from a polycycloalkane. The polycycloalkane may include a C7 to C12 polycycloalkane. For example, the polycycloalkane may include adamantane, norbornane, isobornane, tricyclodecane, tetracyclododecane, or the like.

An aromatic hydrocarbon group as a bivalent hydrocarbon group for Va¹ is a hydrocarbon group having an aromatic ring.

The aromatic hydrocarbon group may include a C3 to C30 aromatic hydrocarbon group. For example, the aromatic hydrocarbon group may include a C5 to C30 aromatic hydrocarbon group. Here, the number of carbon atoms set forth above does not include the number of carbon atoms in a substituent.

The aromatic ring of the aromatic hydrocarbon group may include, for example: an aromatic hydrocarbon ring, such as benzene, biphenyl, fluorene, naphthalene, anthracene, or phenanthrene; an aromatic heterocycle in which some of carbon atoms constituting an aromatic hydrocarbon ring are substituted with heteroatoms; or the like. The heteroatoms in the aromatic heterocycle may include oxygen atoms, sulfur atoms, nitrogen atoms, or the like.

The aromatic hydrocarbon group may include, for example, a group (e.g., an arylene group) obtained by removing two hydrogen atoms from an aromatic hydrocarbon ring, a group (for example, a group obtained by further removing one hydrogen atom from an aryl group of an arylalkyl group, such as a benzyl group, a phenethyl group, a 1-naphthylmethyl group, a 2-naphthylmethyl group, a 1-naphthylethyl group, or a 2-naphthylethyl group) in which one of hydrogen atoms of a group (e.g., an aryl group) obtained by removing one hydrogen atom from an aromatic hydrocarbon ring is substituted with an alkylene group, or the like. In some implementations, the alkylene group (the alkyl chain in the arylalkyl group) has 1 to 4 carbon atoms, for example, 1 or 2 carbon atoms. For example, the alkylene group (the alkyl chain in the arylalkyl group) may have one carbon atom.

In Formula (L1-1), Ra¹ is an acid-labile group represented by Formula (L-r-1) or Formula (L-r-2).

In Formula (L1-2), the (nₐ₂+1)-valent hydrocarbon group for Wa¹ may include an aliphatic hydrocarbon group or an aromatic hydrocarbon group. The aliphatic hydrocarbon group refers to a hydrocarbon group having no aromaticity and may be saturated or unsaturated. For example, the aliphatic hydrocarbon group includes a saturated aliphatic hydrocarbon group. The aliphatic hydrocarbon group may include a linear or branched aliphatic hydrocarbon group, an aliphatic hydrocarbon group including a ring in the structure thereof, or a group obtained by a combination between a linear or branched aliphatic hydrocarbon group and an aliphatic hydrocarbon group including a ring in the structure thereof.

In some implementations, nₐ₂+1 is 2 to 4. For example, nₐ₂+1 may be 2 or 3.

In Formula (L1-2), Ra² is an acid-labile group represented by Formula (L-r-1) or Formula (L-r-3).

Examples of the structural unit represented by Formula (L1-1) are shown below. In the following examples, each Rα independently represents a hydrogen atom, a methyl group, or a trifluoromethyl group.

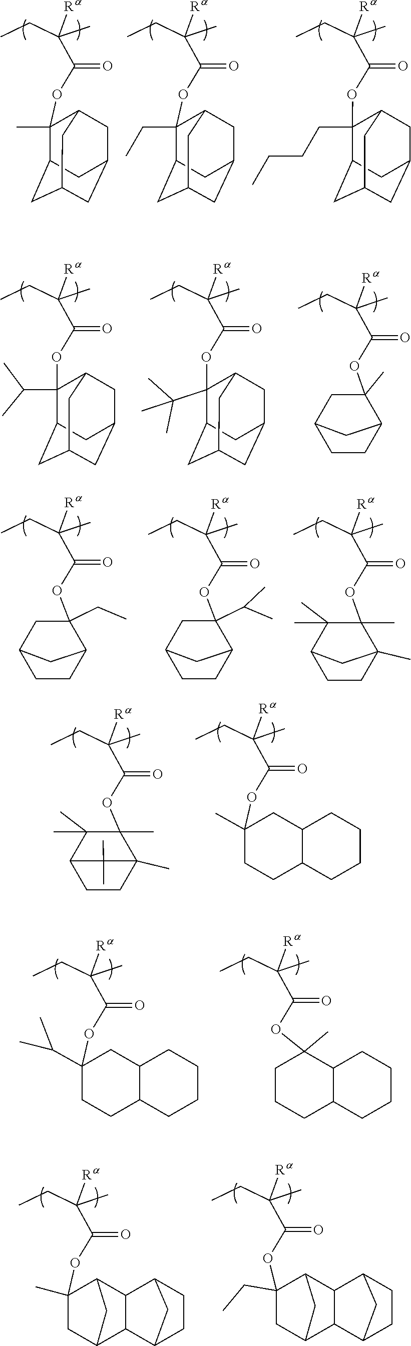

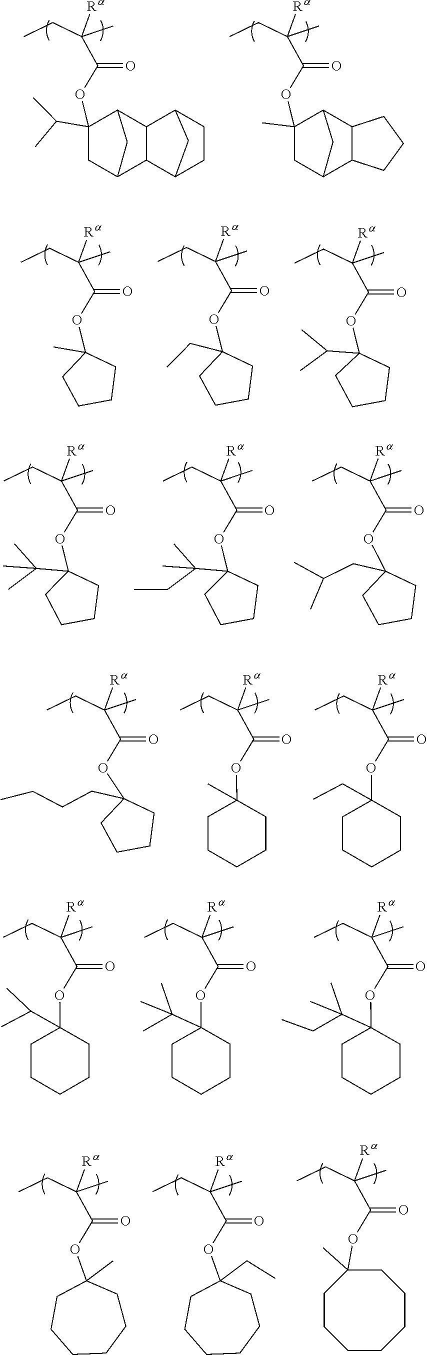

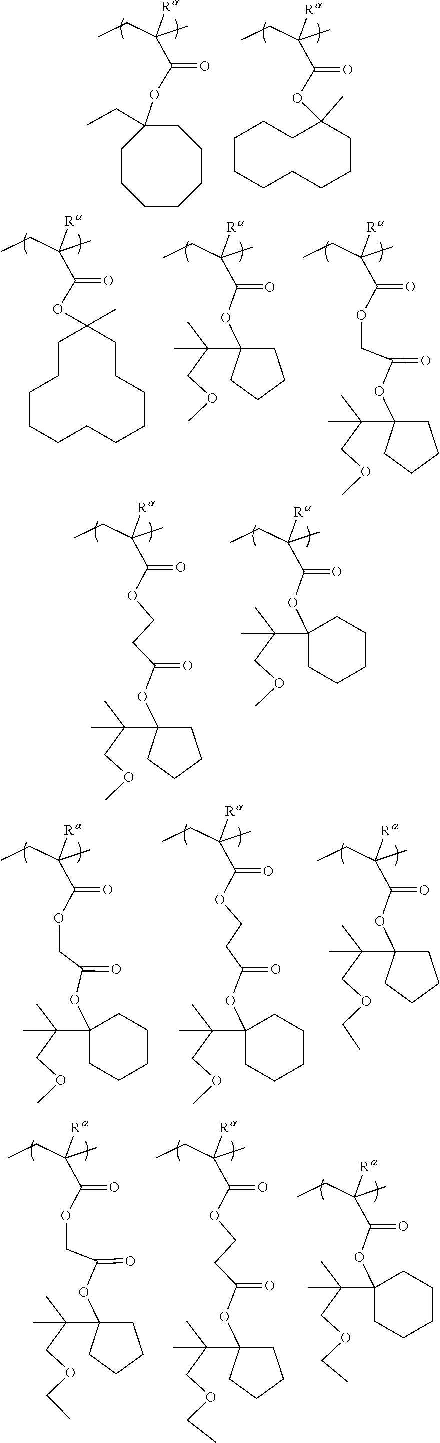

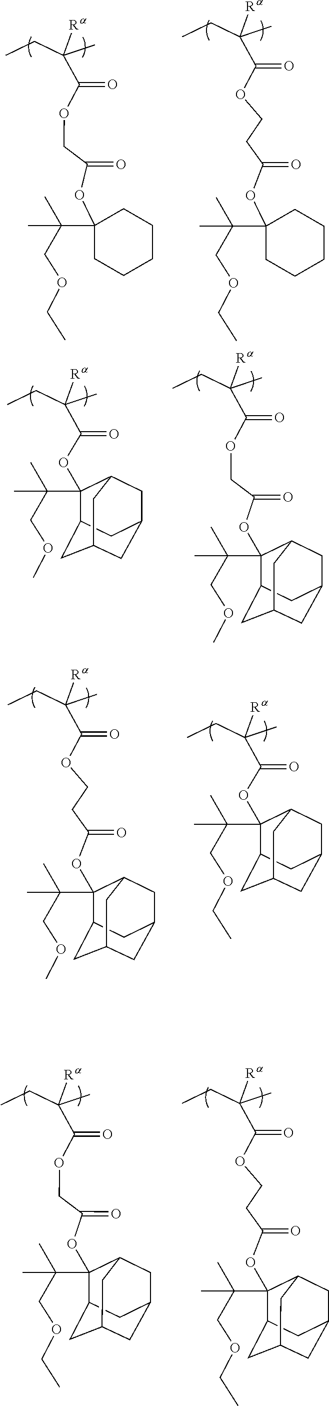

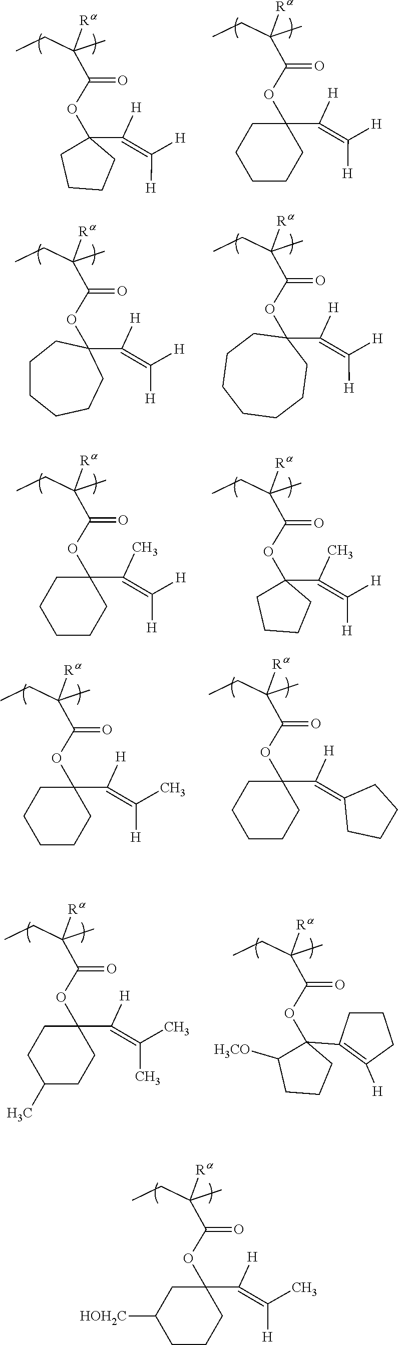

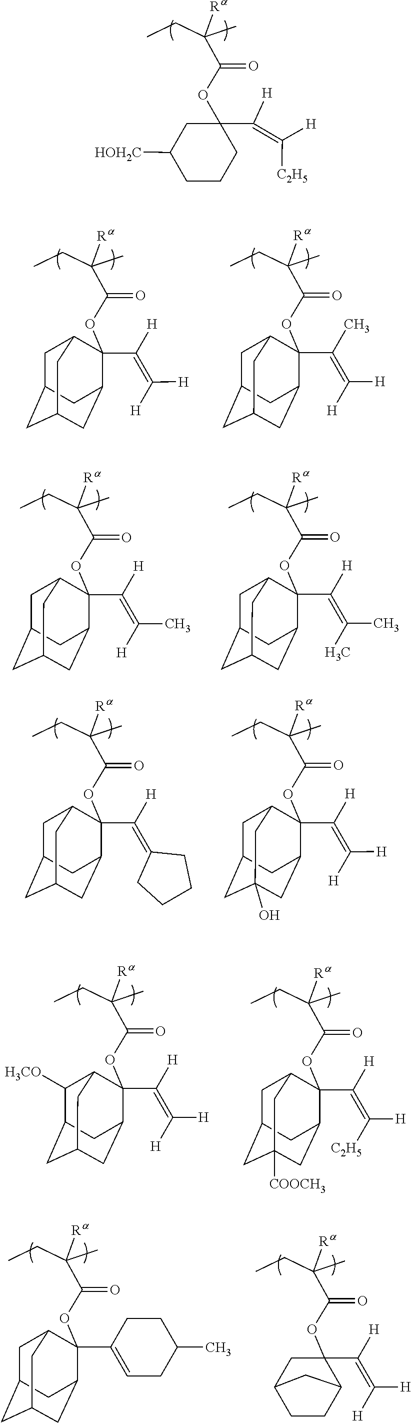

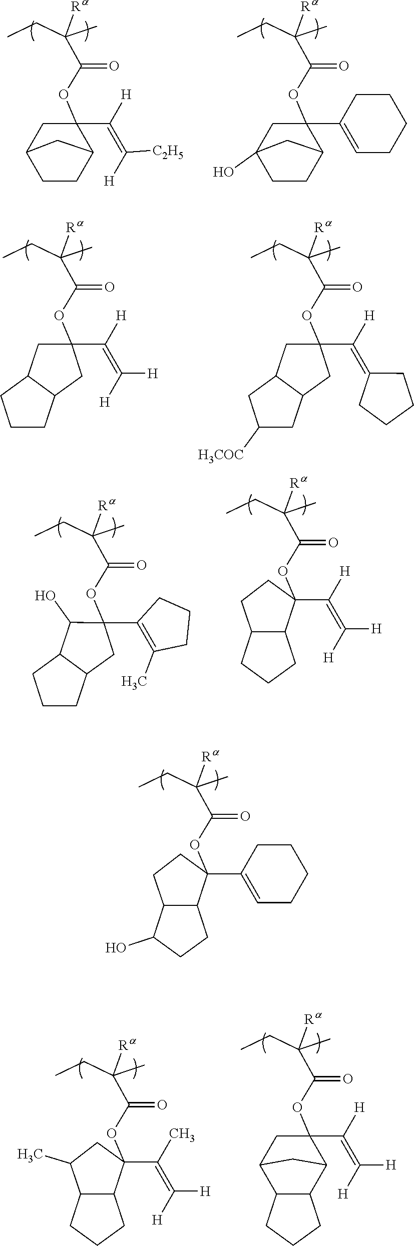

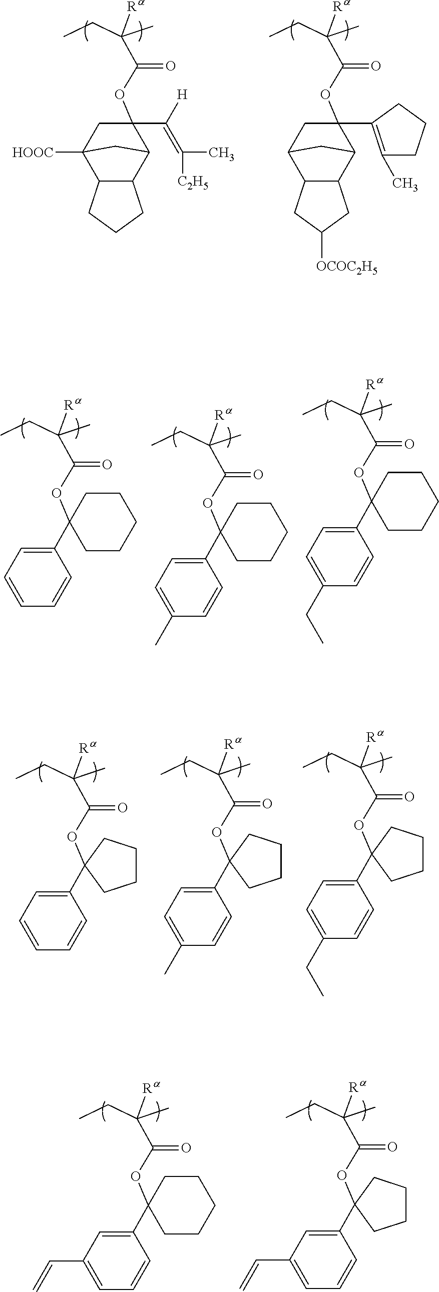

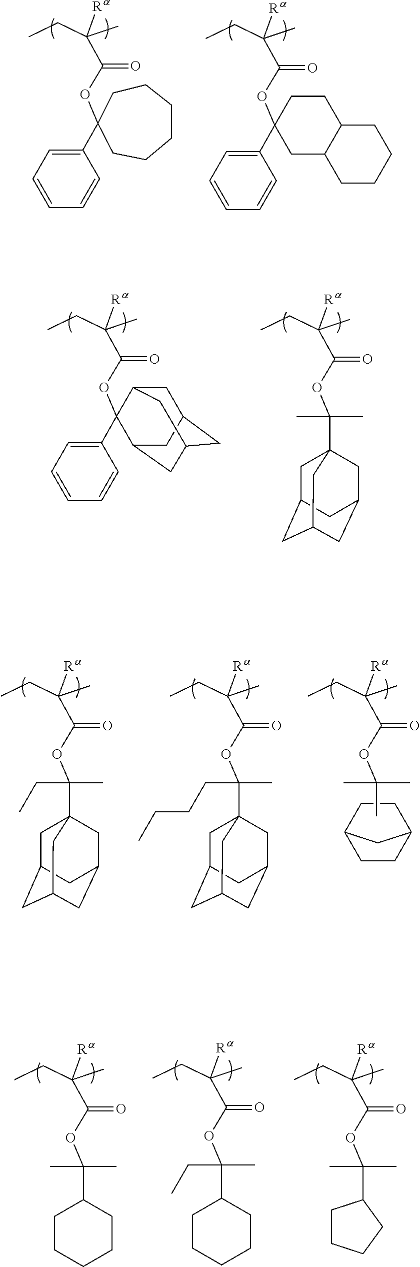

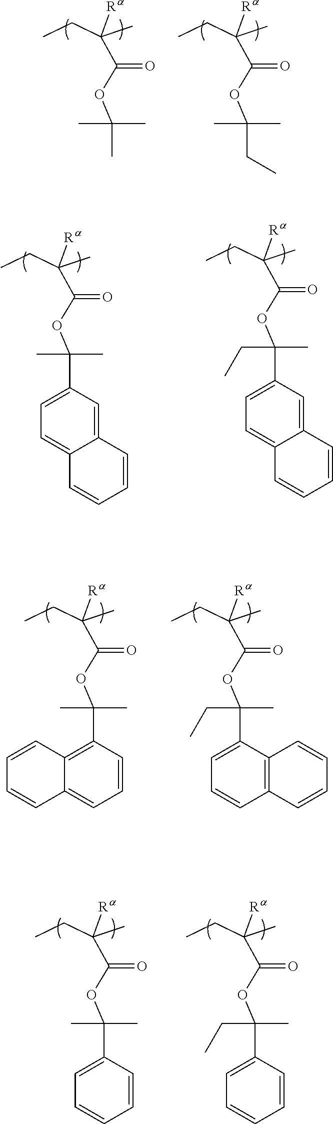

The structural unit (L) of the component (A) may be used alone or in a combination of two or more structural units L.

In some implementations, the structural unit (L) includes a structural unit represented by Formula (L1-1), which can provide improved lithographic characteristics (sensitivity, shapes, and the like). For example, the structural unit (L) may include a structural unit represented by Formula (L1-1-1) shown below.

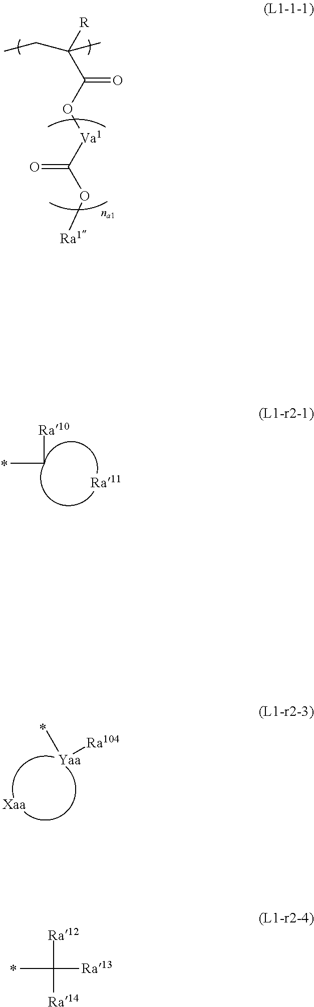

In Formula (L1-1-1), Ra^{1″} is an acid-labile group represented by Formula (L1-r2-1), Formula (L1-r2-3), or Formula (L1-r2-4). In Formula (L1-1-1), R, Va¹, and nₐ₁ are respectively identical to R, Va¹, and nₐ₁ in Formula (L-1).

Descriptions of the acid-labile group represented by Formula (L1-r2-1), Formula (L1-r2-3), or Formula (L1-r2-4) are the same as made above. In some implementations, to improve reactivity, the acid-labile group includes a cyclic group.

In some implementations, in Formula (L1-1-1), Ra^{1″} is an acid-labile group represented by Formula (L1-r2-1). The component (A) may further include a structural unit (which may be referred to as a third structural unit, hereinafter) derived from a polymerizable compound having an ether bond. The polymerizable compound having an ether bond may include, for example, a radical-polymerizable compound, such as a (meth)acrylic acid derivative having an ether bond and an ester bond. The polymerizable compound having an ether bond may include, for example, 2-methoxyethyl (meth)acrylate, 2-ethoxyethyl (meth)acrylate, methoxytriethylene glycol (meth)acrylate, 3-methoxybutyl (meth)acrylate, ethylcarbitol (meth)acrylate, phenoxypolyethylene glycol (meth)acrylate, methoxypolyethylene glycol (meth)acrylate, methoxypolypropylene glycol (meth)acrylate, tetrahydrofurfuryl (meth)acrylate, or the like. These polymerizable compounds having an ether bond may be used alone or in combination.

In addition, the polymeric compound (A) may include a structural unit derived from another polymerizable compound to appropriately control physical and chemical properties of a resist composition. The physical and chemical properties may include (1) solubility in coating solvents, (2) film-formability (glass transition point), (3) alkaline-developability, (4) film loss (selection from among a hydrophilic group, a hydrophobic group, and an alkaline-soluble group), (5) adhesion of a non-light-exposed region with respect to a substrate, (6) etch resistance, and the like.

The other polymerizable compound may include a radical-polymerizable compound or an anionic polymerizable compound, publicly known in the art.

Examples of the other polymerizable compound include: monocarboxylic acids, such as acrylic acid, methacrylic acid, and crotonic acid; dicarboxylic acids, such as maleic acid, fumaric acid, and itaconic acid; methacrylic acid derivatives having a carboxyl group and an ester bond, such as 2-methacryloyloxyethyl succinate, 2-methacryloyloxyethyl maleate, 2-methacryloyloxyethyl phthalate, and 2-methacryloyloxyethyl hexahydrophthalate; (meth)acrylic acid alkyl esters, such as methyl (meth)acrylate, ethyl (meth)acrylate, n-butyl (meth)acrylate, and cyclohexyl (meth)acrylate; (meth)acrylic acid hydroxyalkyl esters, such as 2-hydroxyethyl (meth)acrylate and 2-hydroxypropyl (meth)acrylate; (meth)acrylic acid aryl esters, such as phenyl (meth)acrylate and benzyl (meth)acrylate; dicarboxylic acid diesters, such as diethyl maleate and dibutyl fumarate; vinyl group-containing aromatic compounds, such as styrene, α-methylstyrene, chlorostyrene, chloromethylstyrene, vinyltoluene, hydroxystyrene, α-methylhydroxystyrene, and α-ethylhydroxystyrene; vinyl group-containing aliphatic compounds, such as vinyl acetate; conjugated diolefins, such as butadiene and isoprene; nitrile group-containing polymerizable compounds, such as acrylonitrile and methacrylonitrile; chlorine-containing polymerizable compounds, such as vinyl chloride and vinylidene chloride; amide bond-containing polymerizable compounds, such as acrylamide and methacrylamide; or the like. These other polymerizable compounds may be used alone or in combination.

The component (A) may include a structural unit (P) (which may be referred to as a fourth structural unit, hereinafter) including an acid-nonlabile alicyclic group. The component (A) includes the structural unit (P), thereby improving the etch resistance of a resist pattern that is formed. In addition, the hydrophobicity of the component (A) improves. The improvement in the hydrophobicity of the component (A) contributes to improvements in resolution, resist pattern shapes, and the like, particularly in the case of a solvent development process.

The “acid-nonlabile alicyclic group” in the structural unit (P) is an alicyclic group that is not dissociated even by the action of an acid and intactly remains in the structural unit (P), when the acid is generated in a resist composition by light-exposure (for example, when the acid is generated from an acid-generating structural unit or a component (B) by light-exposure).

The structural unit (P) may include, for example, a structural unit derived from an acrylic acid ester including an acid-nonlabile alicyclic group. The alicyclic group may include a plurality of alicyclic groups, which are known in the art and used for resin components of resist compositions for ArF excimer lasers, KrF excimer lasers, and the like (for example, for ArF excimer lasers).

In some implementations, the alicyclic group includes at least one selected from the group consisting of a tricyclodecyl group, an adamantyl group, a tetracyclododecyl group, an isobornyl group, and a norbornyl group, based on ease of industrial availability. These polycyclic groups may each have a C1 to C5 linear or branched alkyl group as a substituent.

The structural unit (P) of the component (A) may be used alone or in combination of two or more structural units (P).

The amount of the structural unit (O) in the component (A) is not particularly limited, and the structural unit (O) may be present in an amount of about 0.1 mol % to about 30 mol %, for example, about 0.5 mol % to about 20 mol %, or about 1 mol % to about 10 mol %, based on the total amount, 100 mol %, of all structural units of the component (A). Within the above ranges of the amount of the structural unit (O), an acid that is a catalyst may be generated in a sufficient amount, and the sensitivity of a resist composition may improve. In addition, the transmission of light that is used for exposure is sufficient, and thus, excellent resolution and pattern formability may be achieved.

Furthermore, the structural unit (L) (the structural unit having an acid-decomposable group) of the component (A) may be present in an amount of about 20 mol % to about 80 mol %, for example, about 25 mol % to about 75 mol %, or about 30 mol % to about 70 mol %, based on the total amount, 100 mol %, of all structural units of the component (A).

The amount (the molar ratio) of each structural unit in the component (A) may be calculated by, for example, analysis using ¹³C-NMR.

Although the weight-average molecular weight (that is, Mw) of the component (A) is not particularly limited, the component (A) may have a weight-average molecular weight (that is, Mw) of about 1,000 to about 500,000 or about 3,000 to about 100,000. Within the above range of the weight-average molecular weight (that is, Mw) of the component (A), a resist composition may have improved etch resistance and may secure a difference between a dissolution rate before light-exposure and a dissolution rate after light-exposure, thereby securing the resolution thereof. In addition, the dispersity (weight-average molecular weight/number-average molecular weight (that is, Mw/Mn)) of the component (A) is not particularly limited but may range from about 1.0 to about 5.0 or from about 1.0 to about 3.0. The weight-average molecular weight and the dispersity may be measured by gel permeation chromatography (GPC) using conversion based on polystyrene, for example, as described below.

The component (A) may be prepared by a copolymerization reaction of a compound represented by General Formula 1 with, according to the need of the particular structure of the component (A), a polymerizable compound capable of introducing the structural unit (L) or another polymerizable compound. Although there may be several examples of the copolymerization reaction, the copolymerization reaction may include, for example, radical polymerization, anionic polymerization, or coordination polymerization.

For example, the radical polymerization may be performed under, but is not limited to, the following reaction conditions:
(a) as a solvent, hydrocarbons such as benzene, ethers such as tetrahydrofuran, alcohols such as ethanol, or ketones such as methyl ethyl ketone may be used;
(b) as a polymerization initiator, an azo compound such as 2,2′-azobisisobutyronitrile or dimethyl 2,2′-azobis(2-methylpropionate), or peroxide such as benzoyl peroxide or lauroyl peroxide may be used;
(c) the reaction temperature may be maintained at about 0° C. to about 100° C.; and
(d) the reaction time may be about 0.5 hours to about 48 hours.

For example, the anionic polymerization may be performed under, but is not limited to, the following reaction conditions:
(a) as a solvent, hydrocarbons such as benzene, ethers such as tetrahydrofuran, or liquid ammonia may be used;
(b) as a polymerization initiator, a metal such as sodium or potassium, an alkyl metal such as n-butyllithium or sec-butyllithium, a ketyl, or a Grignard reagent may be used;
(c) the reaction temperature may be maintained at about −78° C. to about 0° C.;
(d) the reaction time may be about 0.5 hours to about 48 hours; and
(e) as a terminating agent, a proton-donating compound such as methanol, a halide such as methyl iodide, or other electrophilic reagents may be used.

For example, the coordination polymerization may be performed under, but is not limited to, the following reaction conditions:
(a) as a solvent, hydrocarbons such as n-heptane and toluene may be used;
(b) as a catalyst, a Ziegler-Natta catalyst including alkylaluminum and a transition metal such as titanium, a Phillips catalyst in which chromium and nickel compounds are supported on a metal oxide, an Olefin Metathesis mixed catalyst represented by a tungsten-and-rhenium mixed catalyst, or the like may be used;
(c) the reaction temperature may be maintained at about 0° C. to about 100° C.; and
(d) the reaction time may be about 0.5 hours to about 48 hours.

In addition, a polymeric compound, in which the acid-labile group of the component (A) prepared by the above polymerization method is deprotected in part or in whole, may be used for a negative resist composition. Furthermore, an acid-labile group may be introduced again into the polymeric compound in which the acid-labile group is deprotected, and a substituent different from the acid-labile group introduced during the polymerization may be introduced.

### [Resist Composition]

According to some aspects of the present disclosure, there is provided a resist composition (sometimes referred to hereafter as “the resist composition”) including, at least, the polymeric compound (A) described above and an organic solvent (E) (which may be simply referred to as a component (E), hereinafter). As described above, the polymeric compound (A) includes a structural unit derived from the compound represented by General Formula 1 and a structural unit having a photoacid generating group that generates an acid due to irradiation with active rays or radiation. The resist composition, which includes the polymeric compound (A), may have improved sensitivity and resolution.

When a resist film is formed by using the resist composition and selective light-exposure is performed on the resist film, an acid is generated in a light-exposed region of the resist film, and the solubility of the component (A) in a developer changes due to the action of the acid, whereas, because the solubility of the component (A) in the developer does not change in a non-light-exposed region of the resist film, a difference in solubility in the developer between the light-exposed region and the non-light-exposed region of the resist film is generated. Thus, by developing the resist film, when the resist composition is of a positive type, the light-exposed region of the resist film is dissolved and removed to form a positive-type resist pattern, and when the resist composition is of a negative type, the non-light-exposed region of the resist film is dissolved and removed to form a negative-type resist pattern.

Herein, the resist composition, which forms a positive-type resist pattern by the dissolution and removal of the light-exposed region of the resist film, is also referred to as a positive resist composition, and the resist composition, which forms a negative-type resist pattern by the dissolution and removal of the non-light-exposed region of the resist film, is also referred to as a negative resist composition. The resist composition may include a positive resist composition or a negative resist composition. In addition, the resist composition may be for an alkaline development process, in which an alkaline developer is used for a development process for forming a resist pattern, or for a solvent development process, in which a developer (organic developer) including an organic solvent is used for a development process.

When the resist composition corresponds to a positive resist composition, the resist composition includes a polymeric compound (A) and an organic solvent (E).

In some implementations, when the resist composition corresponds to a positive resist composition, the resist composition further includes at least one selected from an acid generator (photosensitizer) (B), an acid diffusion inhibitor (C), or an adhesion improver (D).

When the resist composition corresponds to a negative resist composition, the resist composition includes a polymeric compound (A) and an organic solvent (E).

In some implementations, when the resist composition corresponds to a negative resist composition, the resist composition further includes at least one selected from an acid generator (photosensitizer) (B), an acid diffusion inhibitor (C), an adhesion improver (D), or a cross-linking agent (F).

Hereinafter, the acid generator (photosensitizer) (B), the acid diffusion inhibitor (C), the adhesion improver (D), the organic solvent (E), the cross-linking agent (F), and other additives that may be included in the resist composition are described.

### Organic Solvent (E)

The resist composition includes an organic solvent (E) (which may be simply referred to herein as a component (E)).

The component (E) may include any organic solvent able to dissolve the component (A) and other additives. Examples of the organic solvent include: ketones, such as cyclohexanone and methyl-2-n-amyl ketone; alcohols, such as 3-methoxybutanol, 3-methyl-3-methoxybutanol, 1-methoxy-2-propanol, and 1-ethoxy-2-propanol; ethers, such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters, such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones, such as γ-butyrolactone. The component (E) may be used alone or in combination of two or more components (E). For example, the component (E) may include at least one selected from the group consisting of propylene glycol monomethyl ether and propylene glycol monomethyl ether acetate.

The component (E) may be present in an amount of about 70 parts by weight to about 3,000 parts by weight, for example, about 100 parts by weight to about 2,500 parts by weight, based on 100 parts by weight of the component (A). When the resist composition includes two or more components (E), the amount of the component (E) refers to the total amount of the two or more components (E).

### Acid Generator (Photosensitizer) (B)

The component (A), which is included in the resist composition, includes a photoacid generating group in the molecule thereof. Separately from the component (A), the resist composition may further include an acid generator (a photosensitizer) (B) (which may be simply referred to as a component (B), hereinafter). The acid generator (B) may include any compound without limitation as long as the compound generates an acid due to irradiation with light (active rays or the like). The component (B) may be used alone or in combination of two or more components (B).

The component (B) may include, for example, an onium salt compound, a halogen-containing compound, a diazoketone compound, a sulfone compound, a sulfonic acid compound, a sulfonimide compound, a diazomethane compound, or the like. In some implementations, the component (B) includes at least one selected from the group consisting of an onium salt compound and a sulfonimide compound, based on ease of availability and the like.

The onium salt compound may include an iodonium salt, a sulfonium salt, a phosphonium salt, a diazonium salt, a pyridinium salt, or the like. The onium salt compound may include: a diaryliodonium salt, such as diphenyliodonium trifluoromethanesulfonate, diphenyliodonium nonafluorobutanesulfonate, diphenyliodonium heptadecafluorooctanesulfonate, diphenyliodonium p-toluenesulfonate, diphenylIodonium hexafluoroantimonate, diphenyliodonium hexafluorophosphate, diphenyliodonium tris(pentafluoroethyl)trifluorophosphate, diphenyliodonium tetrafluoroborate, diphenyliodonium tetrakis(pentafluorophenyl)borate, or diphenyliodonium tris[(trifluoromethyl)sulfonyl]methanide; a triarylsulfonium salt; or the like. The onium salt compound may include a sulfonium salt in order to further improve the sensitivity and thermal stability of the resist composition. The sulfonium salt may include a triarylsulfonium salt in order to further improve the thermal stability of the resist composition.

The triarylsulfonium salt may include, for example, a sulfonium salt, which has: at least one cation selected from the group consisting of a cation represented by General Formula (b1) shown below, a cation represented by General Formula (b2) shown below, a cation represented by General Formula (b3) shown below, and a cation represented by General Formula (b4) shown below; and an anion having at least one skeleton selected from the group consisting of a tetraphenylborate skeleton, a C1 to C20 alkylsulfonate skeleton, a phenylsulfonate skeleton, a 10-camphorsulfonate skeleton, a C1 to C20 trisalkylsulfonylmethanide skeleton, a tetrafluoroborate skeleton, a hexafluoroantimonate skeleton, and a hexafluorophosphate skeleton.

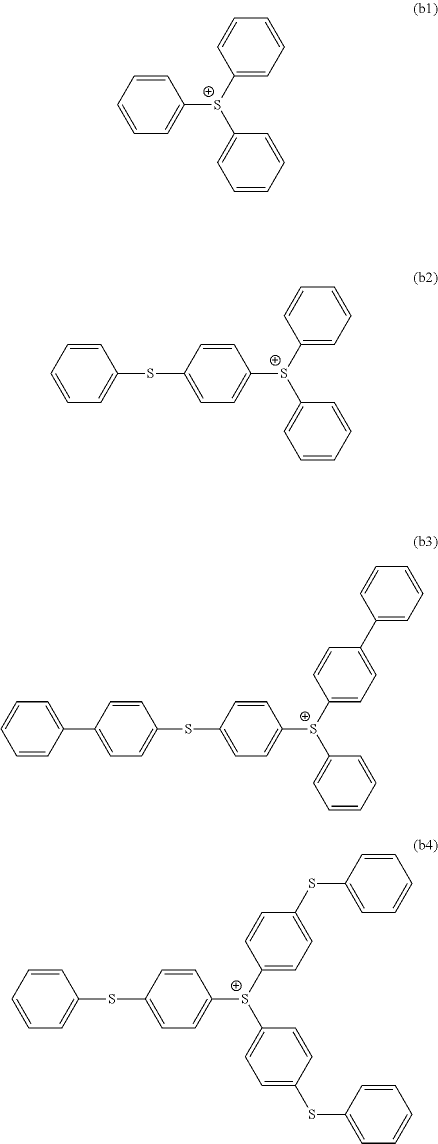 [SMILES: c1ccc([S+](c2ccccc2)c2ccccc2)cc1 ; c1ccc(Sc2ccc([S+](c3ccccc3)c3ccccc3)cc2)cc1 ; c1ccc(-c2ccc(Sc3ccc([S+](c4ccccc4)c4ccc(-c5ccccc5)cc4)cc3)cc2)cc1 ; c1ccc(Sc2ccc([S+](c3ccc(Sc4ccccc4)cc3)c3ccc(Sc4ccccc4)cc3)cc2)cc1]

A hydrogen atom of a phenyl group in Formulae (b1), (b2), (b3), and (b4) may be substituted with a hydroxy group, a C1 to C12 alkyl group, a C1 to C12 alkoxy group, a C2 to C12 alkylcarbonyl group, or a C2 to C12 alkoxycarbonyl group. When there are a plurality of substituents, the plurality of substituents may be identical, or at least some of the plurality of substituents may be different.

A hydrogen atom of a phenyl group of the tetraphenylborate skeleton may be substituted with a fluorine atom, a chlorine atom, a bromine atom, an iodine atom, a cyano group, a nitro group, a hydroxyl group, a C1 to C12 alkyl group, a C1 to C12 alkoxy group, a C2 to C12 alkylcarbonyl group, or a C2 to C12 alkoxycarbonyl group. When there are a plurality of substituents, the plurality of substituents may be identical or different.

A hydrogen atom of the alkylsulfonate skeleton may be substituted with a fluorine atom, a chlorine atom, a bromine atom, an iodine atom, a cyano group, a nitro group, a hydroxyl group, an alkoxy group, an alkylcarbonyl group, or an alkoxycarbonyl group. When there are a plurality of substituents, the plurality of substituents may be identical, or at least some of the plurality of substituents may be different.

A hydrogen atom of a phenyl group of the phenylsulfonate skeleton may be substituted with a fluorine atom, a chlorine atom, a bromine atom, an iodine atom, a cyano group, a nitro group, a hydroxyl group, a C1 to C12 alkyl group, a C1 to C12 alkoxy group, a C2 to C12 alkylcarbonyl group, or a C2 to C12 alkoxycarbonyl group. A plurality of substituents may be identical, or at least some of the plurality of substituents may be different.

A hydrogen atom of the trisalkylsulfonylmethanide skeleton may be substituted with a fluorine atom, a chlorine atom, a bromine atom, an iodine atom, a cyano group, a nitro group, a hydroxyl group, an alkoxy group, an alkylcarbonyl group, or an alkoxycarbonyl group. A plurality of substituents may be identical, or at least some of the plurality of substituents may be different.

A fluorine atom of the hexafluorophosphate may be substituted with a hydrogen atom, a C1 to C12 alkyl group, or a C1 to C12 perfluoroalkyl group. A plurality of substituents may be identical, or at least some of the plurality of substituents may be different.

The sulfonium salt used as the component (B) may include a compound having, as a cation, at least one cation selected from the group consisting of [4-(4-biphenylylthio)phenyl]-4-biphenylylphenylsulfonium, (2-methyl)phenyl[4-(4-biphenylylthio)phenyl]4-biphenylylsulfonium, [4-(4-biphenylylthio)-3-methylphenyl]4-biphenylylphenylsulfonium, (2-ethoxy)phenyl[4-(4-biphenylylthio)-3-ethoxyphenyl]4-biphenylylsulfonium, and tris[4-(4-acetylphenylsulfanyl)phenyl]sulfonium.

The sulfonium salt used as the component (B) may include a compound having, as an anion, at least one anion selected from the group consisting of trifluoromethanesulfonate, nonafluorobutanesulfonate, hexafluoroantimonate, tris[(trifluoromethyl)sulfonyl]methanide, 10-camphorsulfonate, tris(pentafluoroethyl)trifluorophosphate, and tetrakis(pentafluorophenyl)borate.

For example, the sulfonium salt may include, for example, [4-(4-biphenylylthio)phenyl]-4-biphenylylphenylsulfonium trifluoromethanesulfonate, (2-ethoxy)phenyl[4-(4-biphenylylthio)-3-ethoxyphenyl]4-biphenylylsulfonium nonafluorobutanesulfonate, [4-(4-biphenylylthio)phenyl]-4-biphenylylphenylsulfonium tetrakis(pentafluorophenyl)borate, tris[4-(4-acetylphenylsulfanyl)phenyl]sulfonium tetrakis(pentafluorophenyl)borate, or the like.

For example, the sulfonimide compound may include, for example, N-(trifluoromethylsulfonyloxy)succinimide, N-(trifluoromethylsulfonyloxy)phthalimide, N-(trifluoromethylsulfonyloxy)diphenylmaleimide, N-(trifluoromethylsulfonyloxy)bicyclo[2.2.1]hept-5-ene-2,3-dicarboximide, N-(trifluoromethylsulfonyloxy)-1,8-naphthalimide, N-(p-toluenesulfonyloxy)-1,8-naphthalimide, N-(10-camphorsulfonyloxy)-1,8-naphthalimide, or the like.

Although the amount of the component (B) is not particularly limited, the component (B) may be present in an amount of about 0.1 parts by weight to about 15 parts by weight, for example, about 0.3 parts by weight to about 10 parts by weight, or about 0.5 parts by weight to about 5 parts by weight, based on 100 parts by weight of the component (A). When the resist composition includes two or more components (B), the amount of the component (B) refers to the total amount of the two or more components (B).

### Acid Diffusion Inhibitor (C)

The resist composition may include an acid diffusion inhibitor (quencher) (C) (which may be simply referred to as a component (C), hereinafter). The term “acid diffusion inhibitor” is a term widely and generally used in the art and refers to a compound capable of inhibiting a diffusion rate when an acid or the like generated in a system diffuses in a resist film. Due to the addition of the acid diffusion inhibitor (C), it is easy to adjust the resist sensitivity, and the diffusion rate of an acid in a resist film may be inhibited, thereby improving the resolution of the resist composition. In addition, the acid diffusion inhibitor (C) may suppress a sensitivity change after light-exposure and may improve an exposure margin, a pattern profile, or the like by reducing substrate dependence or environment dependence.

The component (C) may include primary, secondary, or tertiary aliphatic amines, mixed amines, aromatic amines, heterocyclic amines, nitrogen-containing compounds having carboxyl groups, nitrogen-containing compounds having sulfonyl groups, nitrogen-containing compounds having hydroxyl groups, nitrogen-containing compounds having hydroxyphenyl groups, alcoholic nitrogen-containing compounds, amides, imides, carbamates, ammonium salts, or the like. The component (C) may be used alone or in combination of two or more components (C).

An aliphatic amine refers to an amine having one or more aliphatic groups, wherein the aliphatic groups may each have 1 to 12 carbon atoms. The aliphatic amine may include an amine (an alkylamine or an alkylalcoholamine) in which at least one of hydrogen atoms of ammonia (NH₃) is substituted with a C1 to C12 alkyl group or a C1 to C12 hydroxyalkyl group, or an alicyclic amine.

Examples of the alkylamine and the alkylalcoholamine may include: monoalkylamines, such as n-hexylamine, n-heptylamine, n-octylamine, n-nonylamine, and n-decylamine; dialkylamines, such as diethylamine, di-n-propylamine, di-n-heptylamine, di-n-octylamine, and dicyclohexylamine; trialkylamines, such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines, such as diethanolamine, triethanolamine, diisopropanolamine, triisopropanolamine, di-n-octanolamine, and tri-n-octanolamine. In some implementations, the alkylamine includes a trialkylamine having a C2 to C10 alkyl group, for example, triethylamine.

The alicyclic amine may include, for example, a heterocyclic compound including a nitrogen atom as a heteroatom. The heterocyclic compound may include a monocyclic compound (an aliphatic monocyclic amine) or a polycyclic compound (an aliphatic polycyclic amine).

The aliphatic monocyclic amine may include, for example, piperidine, piperazine, or the like.

The aliphatic polycyclic amine may include a C6 to C10 aliphatic polycyclic amine, for example, 1,5-diazabicyclo[4.3.0]-5-nonene, 1,8-diazabicyclo[5.4.0]-7-undecene, hexamethylenetetramine, 1,4-diazabicyclo[2.2.2]octane, or the like.

Examples of other aliphatic amines may include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, triethanolamine triacetate, and the like.

Examples of the aromatic amines includes triphenylamine and the like.

Examples of the heterocyclic amines include 4-dimethylaminopyridine, pyrrole, indole, pyrazole, imidazole or derivatives thereof, 2,6-diisopropylaniline, N-tert-butoxycarbonylpyrrolidine, 2,6-di-tert-butylpyridine, and the like.

In addition, as the component (C), the compounds disclosed in the paragraphs [0146] to [0163] of Japanese Patent Publication No. 2008-111103, incorporated herein by reference in its entirety, may also be used.

When the resist composition includes the component (C), although the amount of the component (C) is not particularly limited, the component (C) may be present in an amount of about 0.01 parts by weight to about 5 parts by weight, based on 100 parts by weight of the component (A). Within the above range of the amount of the component (C), a resist pattern shape, delay stability, and the like of the resist composition may improve. In addition, when the resist composition includes two or more components (C), the amount of the component (C) refers to the total amount of the two or more components (C).

### Adhesion Improver (D)

The resist composition may include an adhesion improver (D) (which may be simply referred to as a component (D), hereinafter).

The adhesion improver (D) may include any adhesion improver without limitation as long as the adhesion improver is able to prevent corrosion of a substrate or a metal or the like used for a wiring line and/or improve adhesion to the substrate or the metal or the like. The adhesion improver (D) exhibits a rust-preventive capability by preventing corrosion of a metal. In some implementations, in addition to or instead of such a capability, the adhesion improver (D) may improve adhesion between the resist composition and the substrate or the metal or the like.

The component (D) may include a sulfur-containing compound, an aromatic hydroxy compound, a benzotriazole-based compound, a triazine-based compound, a silicon-containing compound, or the like. The component (D) may be used alone or in combination of two or more components (D).

The sulfur-containing compound may include a compound having a sulfide bond and/or a mercapto group. The sulfur-containing compound may include an acyclic compound or a compound having a cyclic structure.

The acyclic compound may include, for example, dithiodiglycerol [S(CH₂CH(OH)CH₂(OH))₂], bis(2,3-dihydroxypropylthio)ethylene [CH₂CH₂(SCH₂CH(OH)CH₂(OH))₂], sodium 3-(2,3-dihydroxypropylthio)-2-methyl-propylsulfonate [CH₂(OH)CH(OH)CH₂SCH₂CH(CH₃)CH₂SO₃Na], 1-thioglycerol [HSCH₂CH(OH)CH₂(OH)], sodium 3-mercapto-1-propanesulfonate [HSCH₂CH₂CH₂SO₃Na], 2-mercaptoethanol [HSCH₂CH₂(OH)], thioglycolic acid [HSCH₂CO₂H], 3-mercapto-1-propanol [HSCH₂CH₂CH₂(OH)], or the like.

The sulfur-containing compound may include a compound having a sulfide bond and a mercapto group, for example, a heterocyclic compound having a sulfide bond and a mercapto group. In the sulfur-containing compound, the respective numbers of sulfide bonds and mercapto groups are not particularly limited and may each be 1 or more.

The heterocycle may be monocyclic or polycyclic and may include any saturated or unsaturated ring. The heterocycle may additionally include a heteroatom in addition to a sulfur atom. The heteroatom may include an oxygen atom or a nitrogen atom, for example, a nitrogen atom.

The heterocycle may include a C2 to C12 heterocycle, for example, a C2 to C6 heterocycle. The heterocycle may be monocyclic. The heterocycle may include an unsaturated heterocycle. For example, the heterocycle may include an unsaturated and monocyclic heterocycle.

Examples of the heterocycle include the heterocycles shown below.

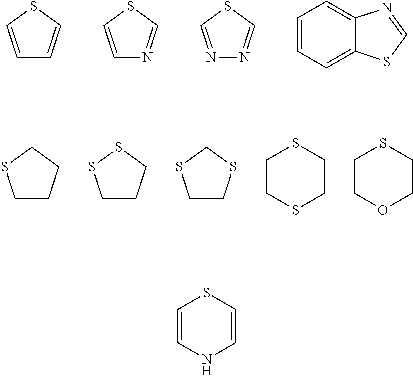

The sulfur-containing compound may include a polymer. The polymer may include a structural unit having a sulfide bond and a mercapto group in a side chain thereof. The structure having a sulfide bond and a mercapto group may be bonded to a main chain via a linkage group, such as an amide bond, an ether bond, a thioether bond, or an ester bond.

The polymer may include a homopolymer or a copolymer. When the polymer includes a copolymer, the polymer may include a structural unit having the acid-labile group described above, a structural unit having no acid-labile group, or the like.

The polymer may have a weight-average molecular weight of 3,000 or more in general, for example, about 5,000 to about 100,000, or about 5,000 to about 50,000. When the sulfur-containing compound includes a polymer, the structural unit having a sulfide bond and a mercapto group may be present in an amount of about 0.1 mol % to about 50 mol %, for example, about 0.5 mol % to about 30 mol %, based on the total amount of all structural units of the polymer for the sulfur-containing compound.

Examples of the sulfur-containing compound may include compounds shown below.

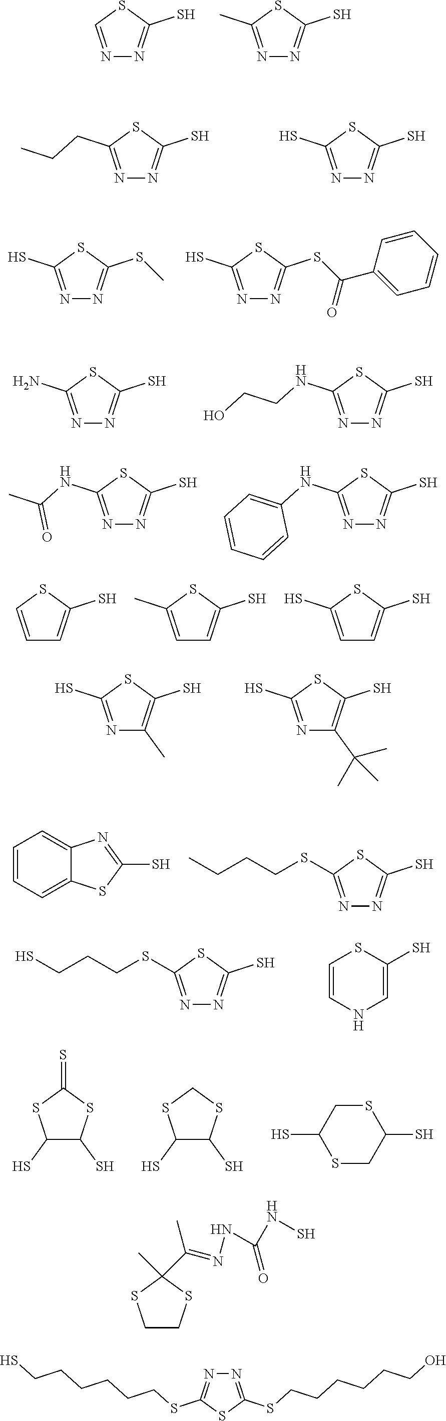

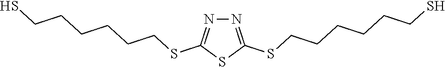 [SMILES: SCCCCCCSc1nnc(SCCCCCCS)s1]

The sulfur-containing compound may be synthesized by a publicly known method (for example, the method disclosed in Japanese Patent Publication No. 2010-79081, incorporated herein by reference in its entirety) or may be commercially available. In addition, the polymer including the sulfur-containing compound may be synthesized by a publicly known method (for example, the method disclosed in Japanese Patent Publication No. 2001-75277, incorporated herein by reference in its entirety) or may be commercially available.

The aromatic hydroxy compound may include, for example, phenol, cresol, xylenol, pyrocatechol (=1,2-dihydroxybenzene), tert-butylcatechol, resorcinol, hydroquinone, pyrogallol, 1,2,4-benzenetriol, salicyl alcohol, p-hydroxybenzyl alcohol, o-hydroxybenzyl alcohol, p-hydroxyphenethyl alcohol, p-aminophenol, m-aminophenol, diaminophenol, aminoresorcinol, p-hydroxybenzoic acid, o-hydroxybenzoic acid, 2,4-dihydroxybenzoic acid, 2,5-dihydroxybenzoic acid, 3,4-dihydroxybenzoic acid, 3,5-dihydroxybenzoic acid, gallic acid, or the like.

The benzotriazole-based compound may include, for example, benzotriazole, 5,6-dimethylbenzotriazole, 1-hydroxybenzotriazole, 1-methylbenzotriazole, 1-aminobenzotriazole, 1-phenylbenzotriazole, 1-hydroxymethylbenzotriazole, methyl 1-benzotriazolecarboxylate, 5-benzotriazolecarboxylic acid, 1-methoxy-benzotriazole, 1-(2,2-dihydroxyethyl)-benzotriazole, 1-(2,3-dihydroxypropyl)benzotriazole, or the like, or may include 2,2′-{[(4-methyl-1H-benzotriazol-1-yl)methyl]imino}bisethanol, 2,2′-{[(5-methyl-1H-benzotriazole-1-yl)methyl]imino}bisethanol, 2,2′-{[(4-methyl-1H-benzotriazol-1-yl)methyl]imino}bisethane, 2,2′-{[(4-methyl-1H-benzotriazol-1-yl)methyl]imino}bispropane, or the like, which is included in the “Irgamet (registered trademark)” series and commercially available from BASF.

The triazine-based compound may include, for example, 1,3,5-triazine-2,4,6-trithiol or the like.

The silicon-containing compound may include, for example, 3-mercaptopropyltrimethoxysilane, 3-mercaptopropyltriethoxysilane, or the like.

When the resist composition includes the component (D), although the amount of the component (D) in the resist composition is not particularly limited, the component (D) may be present in an amount of 0.001 parts by weight or more, for example, 0.002 parts by weight or more, or 0.005 parts by weight or more, based on 100 parts by weight of the component (A). In addition, the component (D) may be present in an amount of 10 parts by weight or less, for example, 3 parts by weight or less, or 1 part by weight or less, based on 100 parts by weight of the component (A). For example, in the resist composition, the component (D) may be present in an amount of about 0.001 parts by weight to about 10 parts by weight, about 0.002 parts by weight to about 3 parts by weight, or about 0.005 parts by weight to about 1 part by weight, based on 100 parts by weight of the component (A). Within the above ranges of the amount of the component (D), the resist composition may form a high-precision resist pattern, and adhesion between a resist pattern and a substrate may be secured. In addition, when the resist composition includes two or more components (D), the amount of the component (D) refers to the total amount of the two or more components (D).

### Cross-Linking Agent (F)

When the resist composition is of a negative type, the resist composition may include a cross-linking agent (F) (which may be simply referred to as a component (F), hereinafter) to reduce the dissolution rate of a light-exposed region, thereby obtaining a negative-type pattern. The cross-linking agent (F) may include an epoxy compound, a melamine compound, a guanamine compound, a glycoluril compound, or a urea compound, which is substituted with at least one group selected from a methylol group, an alkoxymethyl group, and an acyloxymethyl group, or may include an isocyanate compound, an azide compound, a compound including a double bond such as an alkenyloxy group, or the like. In addition, a compound including a hydroxy group may also be used as the cross-linking agent (F). The component (F) may be used alone or in combination of two or more components (F).

The epoxy compound may include tris(2,3-epoxypropyl)isocyanurate, trimethylolmethane triglycidyl ether, trimethylolpropane triglycidyl ether, triethylolethane triglycidyl ether, or the like.

The melamine compound may include hexamethylolmelamine, hexamethoxymethylmelamine, a compound in which 1 to 6 methylol groups of hexamethylolmelamine are methoxymethylated or a mixture thereof, hexamethoxyethylmelamine, hexaacyloxymethylmelamine, a compound in which 1 to 6 methylol groups of hexamethylolmelamine are acyloxymethylated or a mixture thereof, or the like.

The guanamine compound may include tetramethylolguanamine, tetramethoxymethylguanamine, a compound in which 1 to 4 methylol groups of tetramethylolguanamine are methoxymethylated or a mixture thereof, tetramethoxyethylguanamine, tetraacyloxyguanamine, a compound in which 1 to 4 methylol groups of tetramethylolguanamine are acyloxymethylated or a mixture thereof, or the like.

The glycoluril compound may include tetramethylolglycoluril, tetramethoxyglycoluril, tetramethoxymethylglycoluril, a compound in which 1 to 4 methylol groups of tetramethylolglycoluril are methoxymethylated or a mixture thereof, a compound in which 1 to 4 methylol groups of tetramethylolglycoluril are acyloxymethylated or a mixture thereof, or the like. The urea compound may include tetramethylolurea, tetramethoxymethylurea, a compound in which 1 to 4 methylol groups of tetramethylolurea are methoxymethylated or a mixture thereof, tetramethoxyethylurea, or the like.

The isocyanate compound may include tolylene diisocyanate, diphenylmethane diisocyanate, hexamethylene diisocyanate, cyclohexane diisocyanate, or the like.

The azide compound may include 1,1′-biphenyl-4,4′-bisazide, 4,4′-methylidenebisazide, 4,4′-oxybisazide, or the like.

The compound including an alkenyloxy group may include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylolpropane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, trimethylolpropane trivinyl ether, or the like.

When the resist composition is of a negative type and includes the component (F), although the amount of the component (F) is not particularly limited, the component (F) may be present in an amount of about 0.1 parts by weight to about 50 parts by weight, for example, about 1 part by weight to about 40 parts by weight, based on 100 parts by weight of the component (A). When the resist composition includes two or more components (F), the amount of the component (F) refers to the total amount of the two or more components (F).

### Other Additives

The resist composition may include, in addition to or instead of the components/additives described above, other additives, such as a surfactant, a dissolution inhibitor, an acidic compound, a stabilizer, a photosensitizer, a colorant, and the like. In addition, each of the other additives may be present in a general amount within a range not hindering the use of the resist composition for patterning.

### Method of Preparing Resist Composition

The resist composition may be prepared by mixing and stirring the respective components described above by a general method. When mixing and stirring the respective components, a device, such as a dissolver, a homogenizer, or a three-roll mill, may be used. A mixture obtained after uniformly mixing the respective components may be additionally filtered by using a mesh, a membrane filter, or the like.

### Method of Forming Resist Pattern

A method of forming a resist pattern, using the foregoing resist composition, may include a lithography technique publicly known in the art. For example, the method of forming a resist pattern may include a process of forming a resist film on a substrate by using the resist composition, a process of exposing the resist film to light, and a process of forming a resist pattern by developing the resist film after exposure to light.

First, the resist composition is coated on a substrate. The substrate may include a substrate for integrated circuit fabrication (e.g., Si, SiO₂, SiN, SiON, TiN, WSi, BPSG, SOG, an organic anti-reflective film, or a metallic substrate such as copper, chromium, iron, or aluminum), a substrate obtained by forming a certain wiring pattern on the substrate for integrated circuit fabrication, or the like. A material of the wiring pattern may include, for example, copper, aluminum, nickel, gold, or the like.

A coating method may include spin coating, roll coating, flow coating, dip coating, spray coating, doctor coating, or the like. A coating film of the resist composition may have a thickness of, for example, about 0.01 μm to about 2 μm. The coating film undergoes a pre-bake (e.g., a post-apply bake (PAB)) process on a hotplate at a temperature of about 60° C. to about 150° C. for about 10 seconds to about 30 minutes, for example, at a temperature of about 80° C. to about 120° C. for about 30 seconds to about 20 minutes, thereby forming a resist film.

Next, the resist film is exposed to light. Light used for exposure may include high-energy rays, such as ultraviolet light, far-ultraviolet light, electron beams (EBs), extreme ultraviolet (EUV) light having a wavelength of about 3 nm to about 15 nm, X-rays, soft X-rays, excimer laser light, γ-rays, or synchrotron radiation. When ultraviolet light, far-ultraviolet light, EUV light, X-rays, soft X-rays, excimer laser light, γ-rays, or synchrotron radiation is used as the high-energy rays, irradiation is performed directly or by using a mask for forming an intended pattern, at a dose that is at least about 1 mJ/cm² and less than about 700 mJ/cm², for example, at a dose of about 10 mJ/cm² to about 600 mJ/cm². When EBs are used as the high-energy rays, irradiation is performed directly or by using a mask for forming an intended pattern, at a dose of about 0.1 μC/cm² to about 300 μC/cm², for example, about 0.5 μC/cm² to about 200 μC/cm².

An exposure method of the resist film may include immersion exposure. The immersion exposure is an exposure method, in which exposure (immersion exposure) is performed while a space between the resist film and a lens at the lowermost position of an exposure apparatus is filled in advance with a solvent (an immersion medium) having a greater refractive index than that of air. The immersion medium may include a solvent having a refractive index that is greater than the refractive index of air and less than the refractive index of the resist film to be exposed to light, for example, water, a fluorine-based inert liquid, a silicon-based solvent, a hydrocarbon-based solvent, or the like. In some implementations, the immersion medium includes water.

After exposure to light, bake (post-exposure bake (PEB)) may be performed on a hotplate or in an oven at a temperature of about 30° C. to about 150° C. for about 10 seconds to about 30 minutes, for example, at a temperature of about 50° C. to about 120° C. for about 30 seconds to about 20 minutes, or the bake may not be performed.

After exposure to light or the PEB, the resist film undergoes a development process. When the development process is performed, an alkaline developer is used in the case of an alkaline development process, and an organic solvent-containing developer (an organic developer) is used in the case of a solvent development process.

The alkaline developer may include an alkaline aqueous solution of tetramethylammonium hydroxide (TMAH), tetraethylammonium hydroxide, tetrapropylammonium hydroxide, tetrabutylammonium hydroxide, or the like, which has a concentration of about 0.1 wt % to about 10 wt %, for example, about 2 wt % to about 5 wt %.

In the solvent development process, the organic solvent of the organic developer used for the development process may include an organic solvent capable of dissolving the component (A) (the component (A) before exposure to light) and may be suitably selected from among organic solvents publicly known in the art. For example, the organic solvent may include a polar solvent, such as a ketone-based solvent, an ester-based solvent, an alcohol-based solvent, a nitrile-based solvent, am amide-based solvent, or an ether-based solvent, a hydrocarbon-based solvent, or the like. These organic solvents may be used alone or in combination.

The ketone-based solvent is an organic solvent including C—C(═O)—C in the structure thereof. The ester-based solvent is an organic solvent including C—C(═O)—O—C in the structure thereof. The alcohol-based solvent is an organic solvent including an alcoholic hydroxyl group in the structure thereof. The term “alcoholic hydroxyl group” refers to a hydroxyl group bonded to a carbon atom of an aliphatic hydrocarbon group. The nitrile-based solvent is an organic solvent including a nitrile group in the structure thereof. The amide-based solvent is an organic solvent including an amide group in the structure thereof. The ether-based solvent is an organic solvent including C—O—C in the structure thereof.

Among organic solvents, there is an organic solvent including, in the structure thereof, a plurality of functional groups each characterizing each of the aforementioned solvents, and in this case, such an organic solvent is regarded as corresponding to all the solvents respectively including the functional groups of such an organic solvent. For example, diethylene glycol monomethyl ether is regarded as corresponding to both the alcohol-based solvent and the ether-based solvent among the above classifications.

The hydrocarbon-based solvent is a hydrocarbon solvent including a hydrocarbon, which may be halogenated, and having no substituent except for a halogen atom. The halogen atom may include a fluorine atom.

The ketone-based solvent may include, for example, 1-octanone, 2-octanone, 1-nonanone, 2-nonanone, acetone, 4-heptanone, 1-hexanone, 2-hexanone, diisobutyl ketone, cyclohexanone, methylcyclohexanone, phenylacetone, methyl ethyl ketone, methyl isobutyl ketone, acetylacetone, acetonylacetone, ionone, diacetonyl alcohol, acetylcarbinol, acetophenone, methyl naphthyl ketone, isophorone, propylene carbonate, γ-butyrolactone, methyl amyl ketone (2-heptanone), or the like.

The ester-based solvent may include, for example, methyl acetate, butyl acetate, ethyl acetate, isopropyl acetate, amyl acetate, isoamyl acetate, ethyl methoxyacetate, ethyl ethoxyacetate, ethylene glycol monoethyl ether acetate, ethylene glycol monopropyl ether acetate, ethylene glycol monobutyl ether acetate, ethylene glycol monophenyl ether acetate, diethylene glycol monomethyl ether acetate, diethylene glycol monopropyl ether acetate, diethylene glycol monophenyl ether acetate, diethylene glycol monobutyl ether acetate, diethylene glycol monoethyl ether acetate, 2-methoxybutyl acetate, 3-methoxybutyl acetate, 4-methoxybutyl acetate, 3-methyl-3-methoxybutyl acetate, 3-ethyl-3-methoxybutyl acetate, propylene glycol monomethyl ether acetate, propylene glycol monoethyl ether acetate, propylene glycol monopropyl ether acetate, 2-ethoxybutyl acetate, 4-ethoxybutyl acetate, 4-propoxybutyl acetate, 2-methoxypentyl acetate, 3-methoxypentyl acetate, 4-methoxypentyl acetate, 2-methyl-3-methoxypentyl acetate, 3-methyl-3-methoxypentyl acetate, 3-methyl-4-methoxypentyl acetate, 4-methyl-4-methoxypentyl acetate, propylene glycol diacetate, methyl formate, ethyl formate, butyl formate, propyl formate, ethyl lactate, butyl lactate, propyl lactate, ethyl carbonate, propyl carbonate, butyl carbonate, methyl pyruvate, ethyl pyruvate, propyl pyruvate, butyl pyruvate, methyl acetoacetate, ethyl acetoacetate, methyl propionate, ethyl propionate, propyl propionate, isopropyl propionate, methyl 2-hydroxypropionate, ethyl 2-hydroxypropionate, methyl 3-methoxypropionate, ethyl 3-methoxypropionate, ethyl 3-ethoxypropionate, propyl 3-methoxypropionate, or the like.

The nitrile-based solvent may include, for example, acetonitrile, propionitrile, valeronitrile, butyronitrile, or the like.

According to the needs of any specific process, additive(s) publicly known in the art may be added to the organic developer. The additive may include, for example, a surfactant. Although the surfactant is not particularly limited, the surfactant may include, for example, an ionic or nonionic fluorine-based and/or silicon-based surfactant, or the like. When the surfactant is added, the surfactant may be present in an amount of about 0.001 wt % to about 5 wt %, for example, about 0.001 wt % to about 5 wt %, based on the total weight of the organic developer.

The development process may be performed by a development method publicly known in the art. For example, the development method may include a method (a dipping method) in which a substrate is dipped in a developer for a certain time period, a method (a paddle method) in which a developer is stacked on a surface of a substrate by surface tension and then stays for a certain time period, a method (a spray method) in which a developer is sprayed onto a surface of a substrate, or a method (a dynamic dispense method) in which, while a substrate rotating at a certain speed is being scanned at a certain speed by a developer ejection nozzle, a developer continues to be ejected from the developer ejection nozzle onto the substrate.

In the case of a positive resist material, a region irradiated with light is dissolved in a developer, and a region not exposed to light is not dissolved in the developer, thereby forming an intended positive-type pattern on a substrate. In the case of a negative resist material, as opposed to the positive resist material, a region irradiated with light becomes insoluble in a developer, and a region not exposed to light is dissolved in the developer. Although the development time is not particularly limited, the development may be performed for a time period of, for example, about 3 seconds to about 3 minutes.

After the development process, a rinse process may be performed. When the rinse process is performed, water rinse using deionized water may be performed in the case of the alkaline development process, and a rinse solution including an organic solvent may be used in the case of the solvent development process.

In the case of the solvent development process, after the development process or the rinse process, a process of removing the developer or the rinse solution, which is attached on a pattern, by a supercritical fluid may be performed.

As an organic solvent, which is included in the rinse solution used for the rinse process after the development process by the solvent development process, an organic solvent that is unlikely to dissolve a resist pattern may be suitably selected from among the organic solvents listed as examples of the organic solvents used for the organic developer. For example, at least one solvent selected from the hydrocarbon-based solvent, the ketone-based solvent, the ester-based solvent, the alcohol-based solvent, the amide-based solvent, and the ether-based solvent may be used.

When the development is terminated, rinse may be performed. The rinse solution may include a solvent that is compatible with the developer and does not dissolve a resist film. The solvent as such may include a solvent, such as a C3 to C10 alcohol compound, a C8 to C12 ether compound, or a C6 to C12 alkane, alkene, or alkyne, or an aromatic solvent.

The C3 to C10 alcohol compound may include, for example, n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, 1-octanol, or the like.

The C8 to C12 ether compound may include, for example, di-n-butyl ether, diisobutyl ether, di-sec-butyl ether, di-n-pentyl ether, diisopentyl ether, di-sec-pentyl ether, di-tert-pentyl ether, di-n-hexyl ether, or the like.

The C6 to C12 alkane may include, for example, hexane, heptane, octane, nonane, decane, undecane, dodecane, methylcyclopentane, dimethylcyclopentane, cyclohexane, methylcyclohexane, dimethylcyclohexane, cycloheptane, cyclooctane, cyclononane, or the like. The C6 to C12 alkene may include, for example, hexene, heptene, octene, cyclohexene, methylcyclohexene, dimethylcyclohexene, cycloheptene, cyclooctene, or the like. The C6 to C12 alkyne may include, for example, hexyne, heptyne, octyne, or the like.

The aromatic solvent may include, for example, toluene, xylene, ethylbenzene, isopropylbenzene, tert-butylbenzene, mesitylene, or the like.

These organic solvents may be used alone or in combination. In addition, these organic solvents may be used in a mixture with an additional organic solvent or with water. Based on development characteristics, water in the rinse solution may be present in an amount of 30 wt % or less, for example, 10 wt % or less, or 5 wt % or less, based on the total weight of the rinse solution.

According to the needs of any specific process, additive(s) publicly known in the art may be added to the rinse solution. The additive may include, for example, a surfactant. When the surfactant is added, the surfactant may be present in an amount of about 0.001 wt % to about 5 wt %, for example, about 0.005 wt % to about 2 wt %, based on the total weight of the rinse solution.

The rinse process (a cleaning process) using the rinse solution may be performed by a rinse method publicly known in the art. A method for the rinse process may include, for example, a method (a rotating-application method) in which the rinse solution is continues to be ejected onto a substrate rotating at a certain speed, a method (a dipping method) in which a substrate is dipped in the rinse solution for a certain time period, a method (a spray method) in which the rinse solution is sprayed onto a surface of a substrate, or the like. By performing the rinse, the generation of resist pattern collapse or defects may be reduced. In addition, the rinse is not always necessary, and the amount of a solvent used may be reduced by not performing the rinse.

Although various examples have been described above in detail, it will be understood that other examples are also within the scope of the present disclosure.

Compositions and compounds within the scope of the present disclosure include aspects and forms described in the following clauses.

1. A compound represented by General Formula 1:

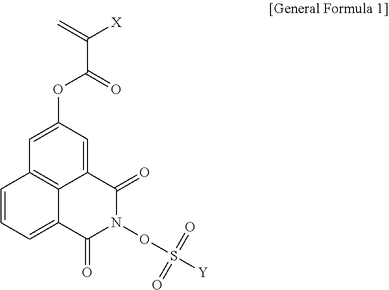

wherein, in General Formula,
X is a hydrogen atom or a methyl group, and
Y is a C1 to C20 linear alkyl group in which at least one hydrogen atom is substituted with a fluorine atom, a C3 to C20 branched alkyl group in which at least one hydrogen atom is substituted with a fluorine atom, an unsubstituted C3 to C20 cycloalkyl group, a C3 to C20 cycloalkyl group in which at least one hydrogen atom is substituted with a fluorine atom, a C3 to C20 cycloalkyl group in which at least one hydrogen atom is substituted with an oxygen atom, an unsubstituted C4 to C21 cycloalkylalkyl group, a C4 to C21 cycloalkylalkyl group in which at least one hydrogen atom is substituted with an oxygen atom, or a C6 to C18 aryl group in which at least one hydrogen atom is substituted with a fluorine atom.

2. The compound of clause 1, wherein Y in General Formula 1 is a C1 to C20 linear alkyl group in which at least one hydrogen atom is substituted with a fluorine atom, a C3 to C20 branched alkyl group in which at least one hydrogen atom is substituted with a fluorine atom, an unsubstituted C3 to C20 cycloalkyl group, a C3 to C20 cycloalkyl group in which at least one hydrogen atom is substituted with a fluorine atom, or a C6 to C18 aryl group in which at least one hydrogen atom is substituted with a fluorine atom.

3. A polymeric compound having a structural unit derived from the compound of clause 1 or 2.

4. The polymeric compound of clause 3, further including a structural unit having an acid-decomposable group.

5. The polymeric compound of clause 3 or 4, wherein the structural unit derived from the compound represented by General Formula 1, in the polymeric compound, is present in an amount of about 0.1 mol % to about 30 mol %, based on the total amount, 100 mol %, of all structural units of the polymeric compound.

6. A resist composition at least including the polymeric compound of any one of clauses 3 to 5 and an organic solvent.

7. The resist composition of clause 6, further including an acid diffusion inhibitor.

8. A method of forming a resist pattern, the method including a process of forming a resist film on a substrate by using the resist composition of clause 6 or 7, a process of exposing the resist film to light, and a process of forming the resist pattern by developing the resist film after exposure to light.

### EXAMPLES

Implementations according to the present disclosure are described in more detail with reference to the following examples and comparative examples, but the scope of the disclosure is not limited to the following examples. In addition, the weight-average molecular weight and the dispersity of a polymeric compound were measured by the following methods.

### Measurement of Weight-Average Molecular Weight and Dispersity

The weight-average molecular weight and the dispersity were measured by GPC and calculated as values converted based on polystyrene.
Measurement apparatus: HLC-8320GPC (Tosoh Corporation)
Column: TSK-GEL SuperAW2500 (three columns connected)
Detector: RI
Mobile phase: Tetrahydrofuran
Column temperature: 40° C.

The weight-average molecular weight (that is, Mw) and the dispersity (that is, Mw/Mn) are values converted based on polystyrene standards (Sigma-Aldrich Co., Ltd.).

### Example 1-1: Synthesis of Compound 1

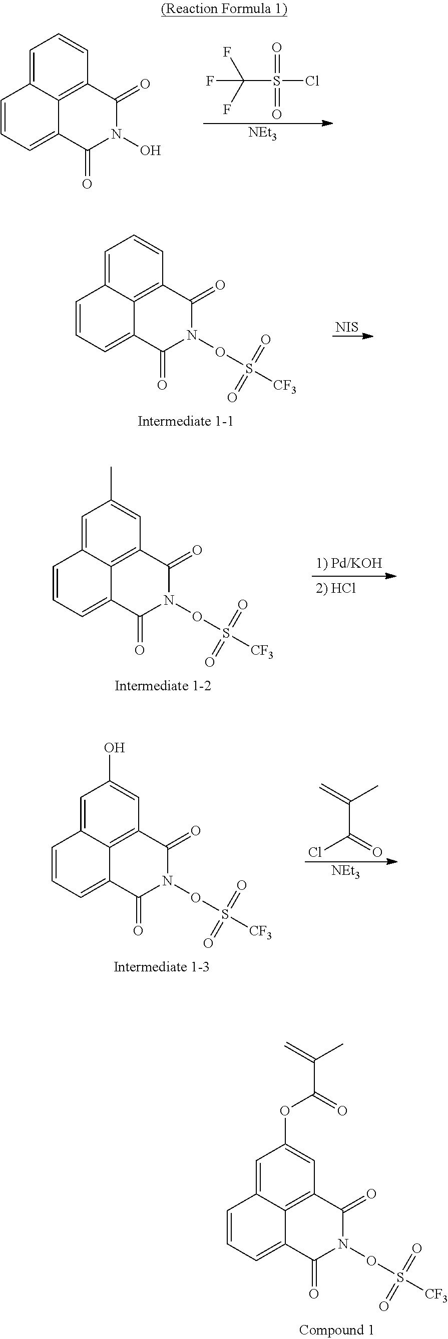

### Synthesis of Intermediate 1-1

20.0 g of N-hydroxynaphthalimide was introduced into a flask and dissolved in 100 g of dichloromethane. Next, 14.2 g of triethylamine was added into the flask, followed by cooling the flask to 0° C., and then, 19.0 g of trifluoromethanesulfonic acid chloride was introduced dropwise into the flask. After the dropwise introduction, the components were reacted at room temperature for 2 hours and then cleaned with deionized water. After the cleaning with deionized water, the reaction solution was concentrated and introduced into methanol, thereby precipitating crystals. The crystals were recovered by filtering and decompression-dried at 40° C., thereby obtaining 30.8 g of Intermediate 1-1 (yield: 95%).

### Synthesis of Intermediate 1-2

25.0 g of Intermediate 1-1 was introduced into a flask and dissolved in 200 g of dichloromethane. Next, 16.5 g of trifluoroacetic acid and 65.2 g of N-iodosuccinimide (NIS) were added into the flask, and the components were reacted at room temperature for 2 hours. The reaction was terminated by dropwise addition of 243 g of a 5 wt % aqueous solution of sodium hydrogen carbonate into the reaction solution, and then, the organic layer was cleaned with deionized water. The organic layer was concentrated and then purified by silica gel column chromatography (developing solvent: dichloromethane/methanol), thereby obtaining 24.6 g of Intermediate 1-2 (yield: 72%).

### Synthesis of Intermediate 1-3

20.0 g of Intermediate 1-2 was introduced into a flask and dissolved in 160 g of N,N-dimethylformamide (DMF), and then, 3.6 g of potassium hydroxide and 1.5 g of tetrakis(triphenylphosphine)palladium were added into the flask. The components were uniformly mixed and then reacted at 120° C. for 2 hours. The reaction solution was cooled to room temperature (25° C.), followed by extracting an intended material by adding 120 g of dichloromethane to the reaction solution, and then, the organic layer was cleaned with 310 g of 1 wt % hydrochloric acid and with deionized water. The organic layer after the cleaning was concentrated and then purified by silica gel column chromatography (developing solvent: dichloromethane/methanol), thereby obtaining 13.3 g of Intermediate 1-3 (yield: 87%).

### Synthesis of Compound 1

15.0 g of Intermediate 1-3, 120 g of dichloromethane, and 10.5 g of triethylamine were introduced into a flask, and 8.7 g of methacrylic acid chloride was added dropwise into the system at 5° C. or less. The components were additionally stirred for 1 hour under ice cooling conditions, followed by performing dropwise addition of 150 g of deionized water into the reaction solution and stirring, and then, the water layer was removed. The organic layer was concentrated and then purified by silica gel column chromatography (developing solvent: dichloromethane/methanol), thereby obtaining 14.6 g of Compound 1 (yield: 82%). ¹H-NMR measurement was performed on Compound 1 that was obtained, and the structure thereof was confirmed by the following data:

¹H-NMR (DMSO-d6, 400 MHz): δ(ppm)=8.61 (d, Ar—H, 1H), 8.54 (d, Ar—H, 1H), 8.26 (s, Ar—H, 1H), 8.09 (s, Ar—H, 1H), 7.79 (t, Ar—H, 1H), 5.88 (d, =CH₂, 1H), 5.70 (d, =CH₂, 1H), 1.90 (s, —CH₃, 3H).

### Example 1-2: Synthesis of Compound 2

12.5 g of Compound 2 (see the following formula) was obtained (yield: 78%) in the same manner as in Example 1-1 except that methacrylic acid chloride in “Synthesis of Compound 1” in Example 1-1 was changed to acrylic acid chloride. ¹H-NMR measurement was performed on Compound 2 that was obtained, and the structure thereof was confirmed by the following data:

¹H-NMR (DMSO-d6, 400 MHz): δ (ppm)=8.58 (d, Ar—H, 1H), 8.52 (d, Ar—H, 1H), 8.25 (s, Ar—H, 1H), 8.10 (s, Ar—H, 1H), 7.78 (t, Ar—H, 1H), 6.40 (d, =CH₂, 1H—), 6.13 (t, ═CH—, 1H), 5.82 (d, =CH₂, 1H).

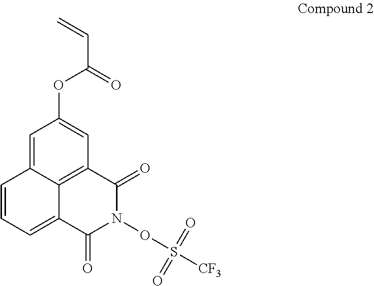 [SMILES: C=CC(=O)Oc1cc2c3c(cccc3c1)C(=O)N(OS(=O)(=O)C(F)(F)F)C2=O]

### Example 1-3: Synthesis of Compound 10

10.9 g of Compound 10 (see the following formula) was obtained (yield: 72%) in the same manner as in Example 1-1 except that trifluoromethanesulfonic acid chloride in “Synthesis of Intermediate 1-1” in Example 1-1 was changed to n-nonafluorobutanesulfonic acid chloride. ¹H-NMR measurement was performed on Compound 10 that was obtained, and the structure thereof was confirmed by the following data:

¹H-NMR (DMSO-d6, 400 MHz): δ (ppm)=8.61 (d, Ar—H, 1H), 8.54 (d, Ar—H, 1H), 8.26 (s, Ar—H, 1H), 8.09 (s, Ar—H, 1H), 7.79 (t, Ar—H, 1H), 5.88 (d, =CH₂, 1H), 5.70 (d, =CH₂, 1H), 1.90 (s, —CH₃, 3H).

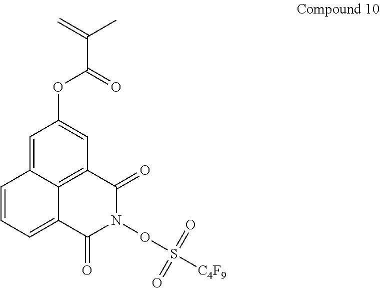

### Example 1-4: Synthesis of Compound 14

11.7 g of Compound 14 (see the following formula) was obtained (yield: 77%) in the same manner as in Example 1-1 except that trifluoromethanesulfonic acid chloride in “Synthesis of Intermediate 1-1” in Example 1-1 was changed to pentafluorobenzenesulfonic acid chloride. ¹H-NMR measurement was performed on Compound 14 that was obtained, and the structure thereof was confirmed by the following data:

¹H-NMR (DMSO-d6, 400 MHz): δ (ppm)=8.63 (d, Ar—H, 1H), 8.52 (d, Ar—H, 1H), 8.28 (s, Ar—H, 1H), 8.13 (s, Ar—H, 1H), 7.80 (t, Ar—H, 1H), 5.86 (d, =CH₂, 1H), 5.68 (d, =CH₂, 1H), 1.89 (s, —CH₃, 3H).

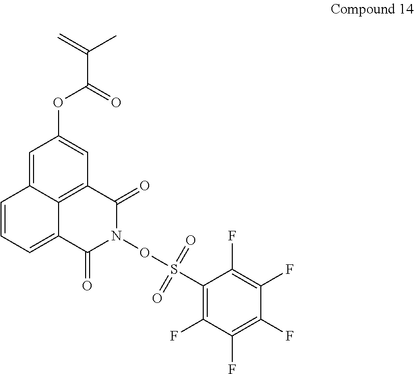 [SMILES: C=C(C)C(=O)Oc1cc2c3c(cccc3c1)C(=O)N(OS(=O)(=O)c1c(F)c(F)c(F)c(F)c1F)C2=O]

### Example 1-5: Synthesis of Compound 21

10.5 g of Compound 21 (see the following formula) was obtained (yield: 75%) in the same manner as in Example 1-1 except that trifluoromethanesulfonic acid chloride in “Synthesis of Intermediate 1-1” in Example 1-1 was changed to norbornylsulfonic acid chloride. ¹H-NMR measurement was performed on Compound 21 that was obtained, and the structure thereof was confirmed by the following data:

¹H-NMR (DMSO-d6, 400 MHz): δ (ppm)=8.60 (d, Ar—H, 1H), 8.55 (d, Ar—H, 1H), 8.24 (s, Ar—H, 1H), 8.10 (s, Ar—H, 1H), 7.78 (t, Ar—H, 1H), 5.89 (d, =CH₂, 1H), 5.73 (d, =CH₂, 1H), 0.99-2.17 (m, 13H).

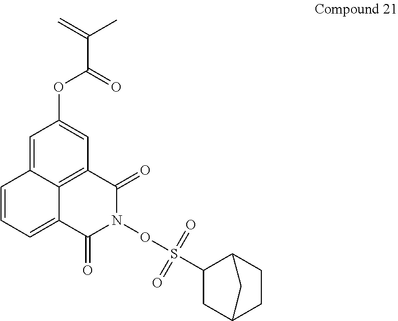 [SMILES: C=C(C)C(=O)Oc1cc2c3c(cccc3c1)C(=O)N(OS(=O)(=O)C1CC3CCC1C3)C2=O]

### Example 1-6: Synthesis of Compound 26

11.6 g of Compound 26 (see the following formula) was obtained (yield: 77%) in the same manner as in Example 1-1 except that trifluoromethanesulfonic acid chloride in “Synthesis of Intermediate 1-1” in Example 1-1 was changed to 10-camphorsulfonic acid chloride. ¹H-NMR measurement was performed on Compound 26 that was obtained, and the structure thereof was confirmed by the following data:

¹H-NMR (DMSO-d6, 400 MHz): δ (ppm)=8.60 (d, Ar—H, 1H), 8.52 (d, Ar—H, 1H), 8.25 (s, Ar—H, 1H), 8.12 (s, Ar—H, 1H), 7.77 (t, Ar—H, 1H), 5.87 (d, =CH₂, 1H—), 5.72 (d, =CH₂, 1H—), 0.72-3.04 (m, 18H).

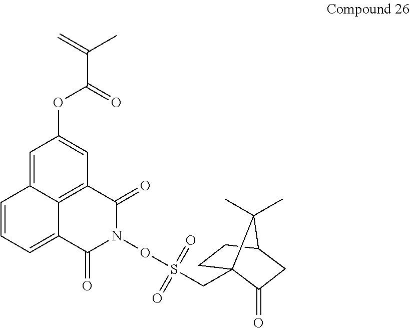 [SMILES: C=C(C)C(=O)Oc1cc2c3c(cccc3c1)C(=O)N(OS(=O)(=O)CC13CCC(CC1=O)C3(C)C)C2=O]

### Comparative Example 1-1: Preparation of Comparative Compound 1

10.3 g of Comparative Compound 1 (see the following formula) was obtained (yield: 75%) in the same manner as in Example 1-1 except that trifluoromethanesulfonic acid chloride in “Synthesis of Intermediate 1-1” in Example 1-1 was changed to methanesulfonyl chloride. ¹H-NMR measurement was performed on Comparative Compound 1 that was obtained, and the structure thereof was confirmed by the following data:

¹H-NMR (DMSO-d6, 400 MHz): δ (ppm)=8.60 (d, Ar—H, 1H), 8.54 (d, Ar—H, 1H), 8.25 (s, Ar—H, 1H), 8.09 (s, Ar—H, 1H), 7.78 (t, Ar—H, 1H), 5.88 (d, =CH₂, 1H), 5.71 (d, =CH₂, 1H), 3.41 (s, —CH₃, 3H), 1.99 (s, —CH₃, 3H).

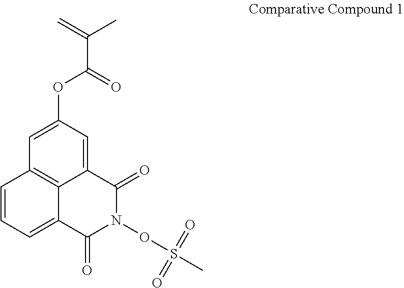 [SMILES: C=C(C)C(=O)Oc1cc2c3c(cccc3c1)C(=O)N(OS(C)(=O)=O)C2=O]

### Comparative Example 1-2: Preparation of Comparative Compound 2

12.0 g of Comparative Compound 2 (see the following formula) was obtained (yield: 72%) in the same manner as in Example 1-1 except that trifluoromethanesulfonic acid chloride in “Synthesis of Intermediate 1-1” in Example 1-1 was changed to p-toluenesulfonyl chloride. ¹H-NMR measurement was performed on Comparative Compound 2 that was obtained, and the structure thereof was confirmed by the following data:

¹H-NMR (DMSO-d6, 400 MHz): δ (ppm)=8.60 (d, Ar—H, 1H), 8.54 (d, Ar—H, 1H), 8.26 (s, Ar—H, 1H), 8.10 (s, Ar—H, 1H), 7.80-7.50 (m, Ar—H, 5H), 5.88 (d, =CH₂, 1H), 5.70 (d, =CH₂, 1H), 2.43 (s, —CH₃, 3H), 1.91 (s, —CH₃, 3H).

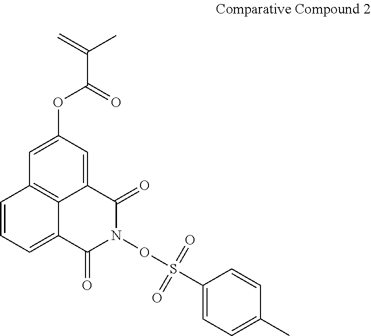 [SMILES: C=C(C)C(=O)Oc1cc2c3c(cccc3c1)C(=O)N(OS(=O)(=O)c1ccc(C)cc1)C2=O]

### Example 2-1: Preparation of Polymer 1

4.0 g of Compound 1 obtained in Example 1-1, 4.0 g of methacrylic acid, 18.55 g of n-butyl methacrylate, 54.9 g of 1-ethylcyclohexyl methacrylate, and 1.9 g of V-601HP (dimethyl-2,2′-azobis(2-methylpropionate), FUJIFILM Wako Pure Chemical Corporation) as a polymerization initiator were dissolved in 165 g of methyl ethyl ketone and stirred at 80° C. for 5 hours in a nitrogen atmosphere. Next, the reaction solution was cooled to room temperature and then reprecipitated by adding the reaction solution to 330 g of heptane. The obtained solids were filtered and decompression-dried at room temperature for one night, thereby obtaining 65.1 g of Polymer 1 as an intended material.

Polymer 1 thus obtained has a weight-average molecular weight (that is, Mw) of 51,000 and a dispersity (that is, Mw/Mn) of 2.5, which were calculated as values converted based on polystyrene standards, through GPC measurement. In addition, the copolymerization composition ratio (molar ratios of the respective structural units in Polymer 1, see the following formula for the structure) of Polymer 1, which was determined by ¹³C-NMR, is 1/m/n/o=60/10/28/2.

### Structural Units of Polymer 1:

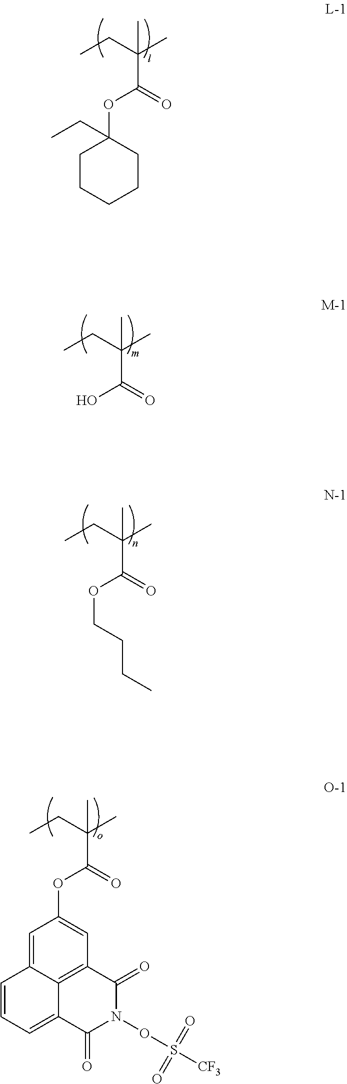

### Examples 2-2, 2-3, and 2-4: Preparation of Polymers 2, 3, and 4

A polymerization reaction was performed in the same manner as in Example 2-1 except that each of Compounds 2, 10, and 14 respectively obtained in Examples 1-2, 1-3, and 1-4 was used instead of Compound 1, thereby obtaining Polymers 2, 3, and 4. The weight-average molecular weight, the dispersity, and the copolymerization composition ratio (molar ratios of the respective structural units in each of Polymers 2, 3, and 4, see the following formulae for the structures thereof) of each of Polymers 2, 3, and 4 that were obtained are shown below in Table 1, the copolymerization composition ratio being determined by ¹³C-NMR.

### Structural Units of Polymer 2:

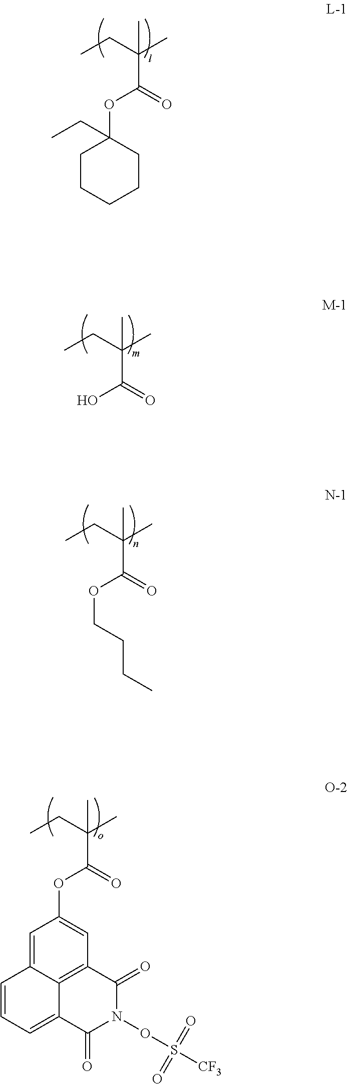

### Structural Units of Polymer 3:

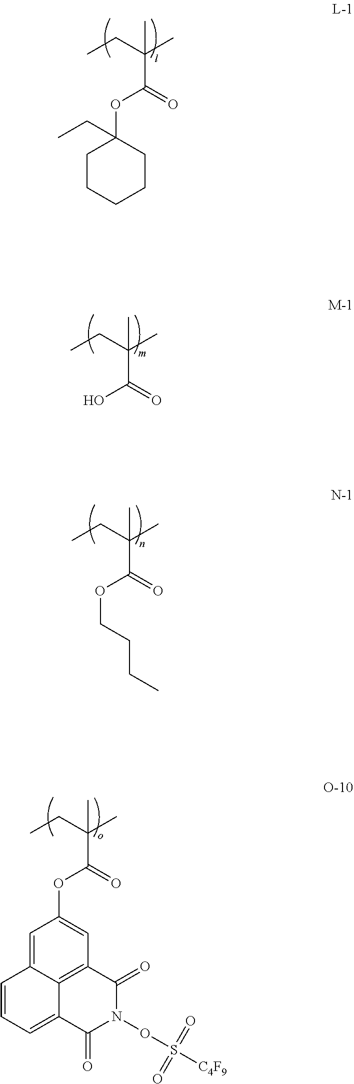

### Structural Units of Polymer 4:

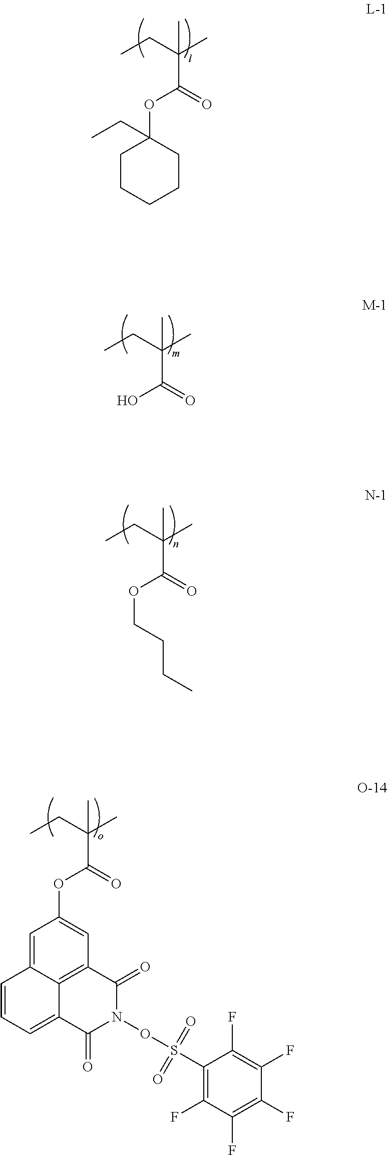

### Example 2-5: Preparation of Polymer 5

A polymerization reaction was performed in the same manner as in Example 2-1 except that 1-ethylcyclopentyl methacrylate was used instead of 1-ethylcyclohexyl methacrylate and Compound 21 obtained in Example 1-5 was used instead of Compound 1, thereby obtaining Polymer 5. The weight-average molecular weight, the dispersity, and the copolymerization composition ratio (molar ratios of the respective structural units in Polymer 5, see the following formulae for the structures thereof) of Polymer 5 that was obtained are shown below in Table 1, the copolymerization composition ratio being determined by ¹³C-NMR.

### Structural Units of Polymer 5:

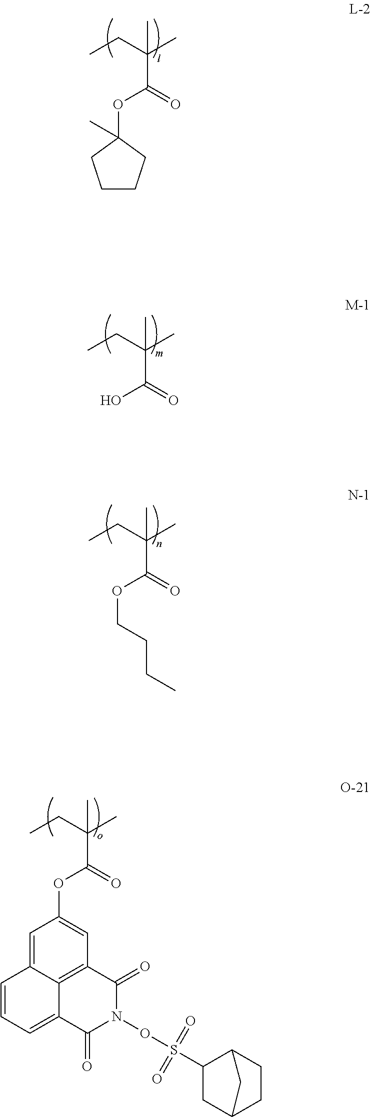

### Example 2-6: Preparation of Polymer 6

A polymerization reaction was performed in the same manner as in Example 2-5 except that Compound 26 obtained in Example 1-6 was used instead of Compound 21, thereby obtaining Polymer 6. The weight-average molecular weight, the dispersity, and the copolymerization composition ratio (molar ratios of the respective structural units in Polymer 6, see the following formulae for the structures thereof) of Polymer 6 that was obtained are shown below in Table 1, the copolymerization composition ratio being determined by ¹³C-NMR.

### Structural Units of Polymer 6:

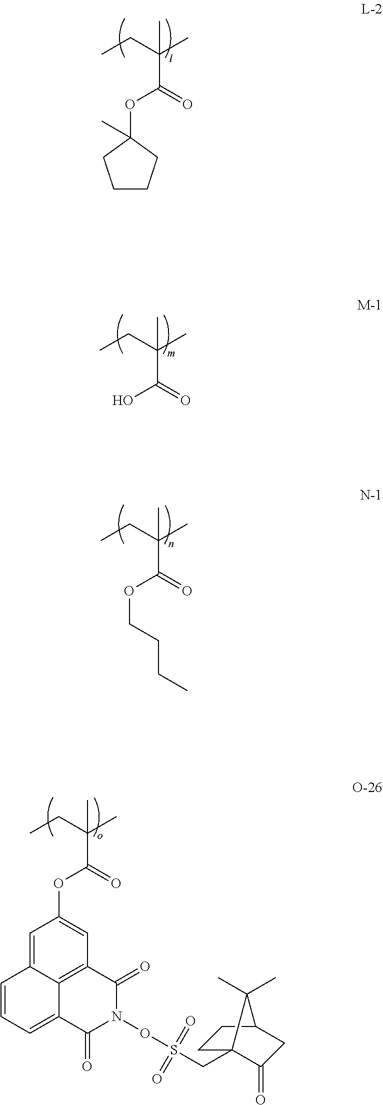

### Example 2-7: Preparation of Polymer 7

A polymerization reaction was performed in the same manner as in Example 2-5 except that Compound 1 obtained in Example 1-1 was used instead of Compound 21, thereby obtaining Polymer 7. The weight-average molecular weight, the dispersity, and the copolymerization composition ratio (molar ratios of the respective structural units in Polymer 7, see the following formulae for the structures thereof) of Polymer 7 that was obtained are shown below in Table 1, the copolymerization composition ratio being determined by ¹³C-NMR.

### Structural Units of Polymer 7:

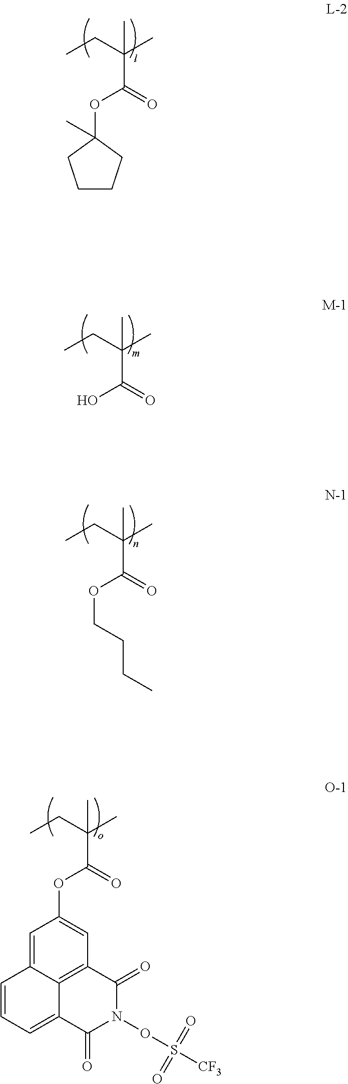

### Comparative Example 2-1: Preparation of Polymer 8

A polymerization reaction was performed in the same manner as in Example 2-5 except that Comparative Compound 1 prepared in Comparative Example 1-1 was used instead of Compound 5, thereby obtaining Polymer 8. The weight-average molecular weight, the dispersity, and the copolymerization composition ratio (molar ratios of the respective structural units in Polymer 8, see the following formulae for the structures thereof) of Polymer 8 that was obtained are shown below in Table 1, the copolymerization composition ratio being determined by ¹³C-NMR.

### Structural Units of Polymer 8:

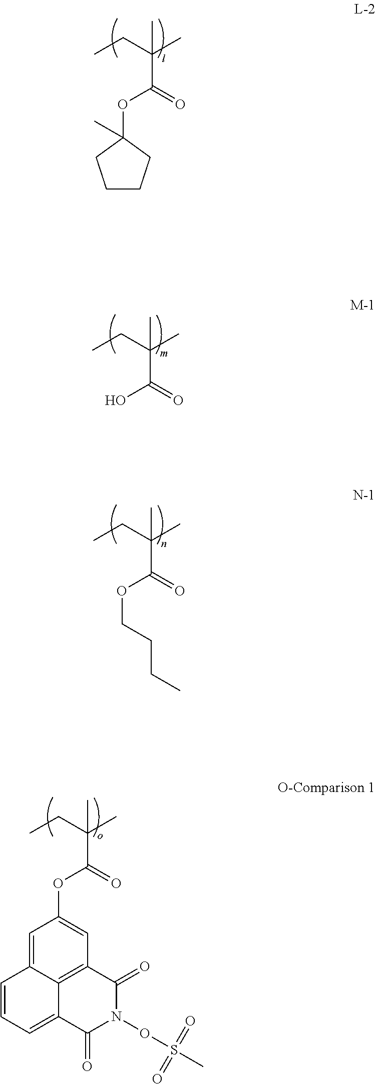

### Comparative Example 2-2: Preparation of Polymer 9

A polymerization reaction was performed in the same manner as in Example 2-5 except that Comparative Compound 2 prepared in Comparative Example 1-2 was used instead of Compound 5, thereby obtaining Polymer 9. The weight-average molecular weight, the dispersity, and the copolymerization composition ratio (molar ratios of the respective structural units in Polymer 9, see the following formulae for the structures thereof) of Polymer 9 that was obtained are shown below in Table 1, the copolymerization composition ratio being determined by ¹³C-NMR.

### Structural Units of Polymer 9:

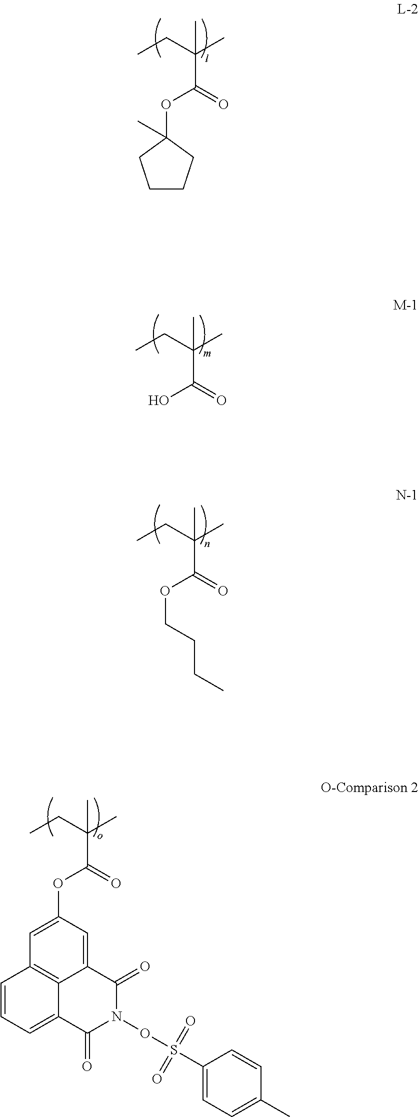

### Comparative Example 2-3: Preparation of Polymer 10

4.0 g of methacrylic acid, 19.8 g of n-butyl methacrylate, 54.7 g of 1-ethylcyclohexyl methacrylate, and 1.9 g of V-601HP (FUJIFILM Wako Pure Chemical Corporation) as a polymerization initiator were dissolved in 165 g of methyl ethyl ketone and stirred at 80° C. for 5 hours in a nitrogen atmosphere. Next, the reaction solution was cooled to room temperature and then reprecipitated by adding the reaction solution to 330 g of heptane. The obtained solids were filtered and decompression-dried at room temperature for one night, thereby obtaining 62.8 g of Polymer 10 that is an intended material.

The weight-average molecular weight, the dispersity, and the copolymerization composition ratio (molar ratios of the respective structural units in Polymer 10, see the following formulae for the structures thereof) of Polymer 10 that was obtained are shown below in Table 1, the copolymerization composition ratio being determined by ¹³C-NMR.

### Structural Units of Polymer 10:

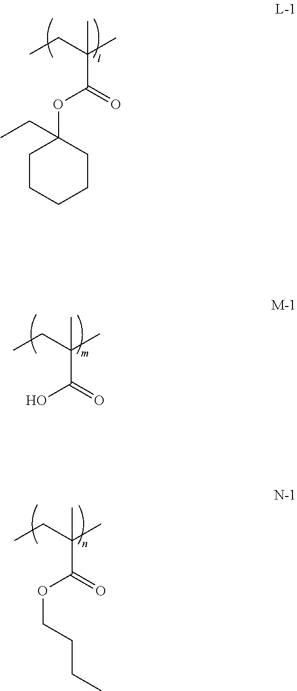

TABLE 1

| | | Types of structural units | | | | | |
|---|---|---|---|---|---|---|---|
| | Polymer No. | Copolymerization composition ratio (molar ratio) | | | | Mw | Mw/Mn |
| Example 2-1 | Polymer 1 | L-1 | M-1 | N-1 | O-1 | 51,000 | 2.5 |
| | | 60 | 10 | 28 | 2 | | |
| Example 2-2 | Polymer 2 | L-1 | M-1 | N-1 | O-2 | 50,000 | 2.4 |
| | | 59 | 10 | 29 | 2 | | |
| Example 2-3 | Polymer 3 | L-1 | M-1 | N-1 | O-10 | 49,000 | 2.3 |
| | | 59 | 10 | 29 | 2 | | |
| Example 2-4 | Polymer 4 | L-1 | M-1 | N-1 | O-14 | 51,000 | 2.6 |
| | | 60 | 10 | 28 | 2 | | |
| Example 2-5 | Polymer 5 | L-2 | M-1 | N-1 | O-21 | 48,000 | 2.3 |
| | | 60 | 10 | 28 | 2 | | |
| Example 2-6 | Polymer 6 | L-2 | M-1 | N-1 | O-26 | 47,000 | 2.4 |
| | | 60 | 10 | 28 | 2 | | |
| Example 2-7 | Polymer 7 | L-2 | M-1 | N-1 | O-1 | 52,000 | 2.5 |
| | | 60 | 10 | 28 | 2 | | |
| Comparative | Polymer 8 | L-2 | M-1 | N-1 | C1* | 49,000 | 2.7 |
| Example 2-1 | | 59 | 11 | 28 | 2 | | |
| Comparative | Polymer 9 | L-2 | M-1 | N-1 | C2* | 50,000 | 2.6 |
| Example 2-2 | | 60 | 11 | 27 | 2 | | |
| Comparative | Polymer 10 | L-1 | M-1 | N-1 | — | 48,000 | 2.4 |
| Example 2-3 | | 61 | 10 | 29 | — | | |

(In Table 1, C1* = O-comparison 1, C2* = O-comparison 2)

### Example 3-1: Preparation of Resist Composition 1

Based on 100 parts by weight of Polymer 1 obtained in Example 2-1, 0.1 parts by weight of triethylamine (Tokyo Chemical Industry Co., Ltd., Component C-1) as an acid diffusion inhibitor, 0.05 parts by weight of 2-mercaptobenzothiazole (Tokyo Chemical Industry Co., Ltd., Component D-1) as an adhesion improver, and 100 parts by weight of a mixed solvent (propylene glycol monomethyl ether acetate (PGMEA):propylene glycol monomethyl ether (PGME)=50:50 (weight ratio), Component E-1) of PGMEA and PGME, as a solvent, were prepared. These components were mixed at 25° C. for 15 minutes, thereby preparing Resist Composition 1.

### Examples 3-2, 3-3, 3-4, 3-5, 3-6, and 3-7: Preparation of Resist Compositions 2 to 7

Resist Compositions 2 to 7 were prepared in the same manner as in Example 3-1 except that Polymers 2 to 7 obtained in Examples 2-2, 2-3, 2-4, 2-5, 2-6, and 2-7 were used instead of Polymer 1, respectively.

### Example 3-8

Resist Composition 8 was prepared in the same manner as in Example 3-1 except that 0.5 parts by weight of N-(trifluoromethylsulfonyloxy)-1,8-naphthalimide (Tokyo Chemical Industry Co., Ltd., Component B-1, see the following formula) was further added based on 100 parts by weight of Polymer 1.

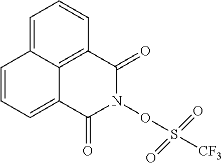 [SMILES: O=C1c2cccc3cccc(c23)C(=O)N1OS(=O)(=O)C(F)(F)F]

### Example 3-9

Resist Composition 9 was prepared in the same manner as in Example 3-1 except that 0.5 parts by weight of [4-(4-biphenylylthio)phenyl]-4-biphenylylphenylsulfonium trifluoromethanesulfonate (San-Apro Ltd., Component B-2, see the following formula) was further added based on 100 parts by weight of Polymer 1.

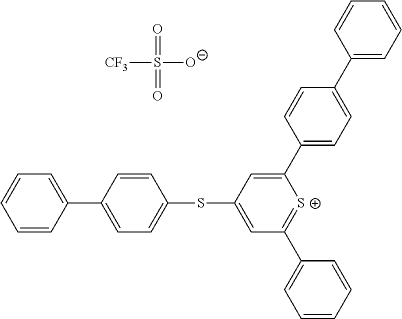 [SMILES: c1ccc(-c2ccc(Sc3cc(-c4ccccc4)[s+]c(-c4ccc(-c5ccccc5)cc4)c3)cc2)cc1 ; O=S(=O)([O-])C(F)(F)F]

### Comparative Examples 3-1 and 3-2

Comparative Resist Compositions 1 and 2 were prepared in the same manner as in Example 3-1 except that Polymers 8 and 9 obtained in Comparative Examples 2-1 and 2-2 were used instead of Polymer 1, respectively.

### Comparative Example 3-3

Comparative Resist Composition 3 was prepared in the same manner as in Example 3-8 except that Polymer 10 obtained in Comparative Example 2-3 was used instead of Polymer 1 and the added amount of Component B-1 was changed to 2 parts by weight based on 100 parts by weight of Polymer 10.

### Comparative Example 3-4

Comparative Resist Composition 4 was prepared in the same manner as in Example 3-9 except that Polymer 10 obtained in Comparative Example 2-3 was used instead of Polymer 1 and the added amount of Component B-2 was changed to 2 parts by weight based on 100 parts by weight of Polymer 10.

### Evaluation

A Cu substrate (size: 8 inch), in which a Cu film with a thickness of 150 nm was formed on a surface of a silicon substrate by sputtering, was prepared. The resist composition of each of Examples and Comparative Examples was coated on the Cu substrate by a spin-coating method, followed by drying the resist composition at 150° C. for 10 minutes by using a hotplate, thereby forming a resist film with a thickness of 60 μm. Next, the resist pattern was exposed through a photomask to i-line (wavelength: 365 nm) at an arbitrary dose by using the i-line stepper, FPA-5520iV (Canon Inc., NA=0.18), the photomask having hole pattern with sizes from 10 μm to 60 m in increments of 5 μm. Next, the Cu substrate was placed on a hotplate and underwent PEB at 100° C. for 5 minutes. Next, an operation of maintaining a 2.38 wt % TMAH aqueous solution on the resist film at 25° C. for 60 seconds (paddle development) was performed six times in total. Next, a resist pattern surface was rinsed at 25° C. for 60 seconds by deionized water and then spin-dried, thereby obtaining a resist pattern.

The obtained resist pattern (hole patterns) was observed by a scanning electron microscope (SEM) (Hitachi, Ltd., model: S9220), and the minimum hole diameter (unit: μm) able to be resolved was evaluated as “resolution”. In addition, the obtained resist pattern (hole patterns) was observed by the SEM, and the minimum exposure dose (unit: mJ/cm²) allowing the 40 μm-hole pattern to be resolved was evaluated as “sensitivity”.

Results of the evaluation are shown below in Table 2.

TABLE 2

| | | | Acid | | | |
|---|---|---|---|---|---|---|
| | Resist | Compound | geverator | | | |
| | Composition | of General | (parts by | Polymeric | Resolution | Sensitivity |
| | No. | Formula 1 | weight) | compound | (μm) | (mJ/cm²) |
| Example 3-1 | 1 | Compound | — | Polymer 1 | 25 | 400 |
| | | 1 | | | | |
| Example 3-2 | 2 | Compound | — | Polymer 2 | 25 | 400 |
| | | 2 | | | | |
| Example 3-3 | 3 | Compound | — | Polymer 3 | 20 | 400 |
| | | 10 | | | | |
| Example 3-4 | 4 | Compound | — | Polymer 4 | 20 | 400 |
| | | 14 | | | | |
| Example 3-5 | 5 | Compound | — | Polymer 5 | 25 | 500 |
| | | 21 | | | | |
| Example 3-6 | 6 | Compound | — | Polymer 6 | 25 | 500 |
| | | 26 | | | | |
| Example 3-7 | 7 | Compound | — | Polymer 7 | 25 | 400 |
| | | 1 | | | | |
| Example 3-8 | 8 | Compound | B-1(0.5) | Polymer 1 | 25 | 400 |
| | | 1 | | | | |
| Example 3-9 | 9 | Compound | B-2(0.5) | Polymer 1 | 25 | 400 |
| | | 1 | | | | |
| Comparative | Comparative | Compound | — | Polymer 8 | 40 | 700 |
| Example 3-1 | 1 | 7 | | | | |
| Comparative | Comparative | Compound | — | Polymer 9 | 35 | 800 |
| Example 3-2 | 2 | 8 | | | | |
| Comparative | Comparative | — | B-1(2) | Polymer | 40 | 800 |
| Example 3-3 | 3 | | | 10 | | |
| Comparative | Comparative | — | B-2(2) | Polymer | 40 | 800 |
| Example 3-4 | 4 | | | 10 | | |

As clearly shown in Table 2, it can be seen that the resist compositions of Examples 3-1, 3-2, 3-3, 3-4, 3-5, 3-6, 3-7, 3-8, and 3-9 have excellent sensitivity and resolution. On the other hand, it can be seen that the resist compositions of Comparative Examples 3-1, 3-2, 3-3, and 3-4 each using a polymeric compound not including a structural unit derived from the compound represented by General Formula 1 have deteriorations in both sensitivity and resolution.

While this disclosure contains many specific implementation details, these should not be construed as limitations on the scope of what may be claimed. Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a combination can in some cases be excised from the combination, and the combination may be directed to a subcombination or variation of a subcombination.

While various examples have been described, it will be understood that various changes in form and details may be made therein without departing from the spirit and scope of this disclosure.

1. A compound represented by General Formula 1: 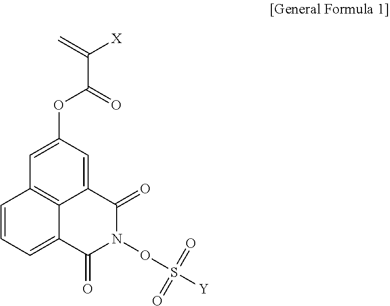
wherein, in General Formula 1,
X is a hydrogen atom or a methyl group, and
Y is: a C1 to C20 linear alkyl group in which at least one hydrogen atom is substituted with a fluorine atom, a C3 to C20 branched alkyl group in which at least one hydrogen atom is substituted with a fluorine atom, an unsubstituted C3 to C20 cycloalkyl group, a C3 to C20 cycloalkyl group in which at least one hydrogen atom is substituted with a fluorine atom, a C3 to C20 cycloalkyl group in which at least one hydrogen atom is substituted with an oxygen atom, an unsubstituted C4 to C21 cycloalkylalkyl group, a C4 to C21 cycloalkylalkyl group in which at least one hydrogen atom is substituted with an oxygen atom, or a C6 to C18 aryl group in which at least one hydrogen atom is substituted with a fluorine atom.

2. The compound of claim 1, wherein, in General Formula 1, Y is the C1 to C20 linear alkyl group in which at least one hydrogen atom is substituted with a fluorine atom, the C3 to C20 branched alkyl group in which at least one hydrogen atom is substituted with a fluorine atom, an unsubstituted C3 to C20 cycloalkyl group, the C3 to C20 cycloalkyl group in which at least one hydrogen atom is substituted with a fluorine atom, or the C6 to C18 aryl group in which at least one hydrogen atom is substituted with a fluorine atom.

3. The compound of claim 1, wherein, in General Formula 1, Y is a C1 to C15 fluoroalkyl group or a C1 to C15 partially fluorinated alkyl group.

4. The compound of claim 1, wherein, in General Formula 1, Y is —CF₃, —C₂F₅, —C₃F₇, —C₄F₉, —C₅F₁₁, —C₆F₁₃, —C₇F₁₅, or —C₈F₁₇.

5. The compound of claim 1, wherein, in General Formula 1, Y is —C₃H₄F₃, —C₄HF₈, or —C₆H₄F₉.

6. The compound of claim 1, wherein, in General Formula 1, Y is a cyclopropyl group, a cyclobutyl group, a cyclopentyl group, a cyclohexyl group, a cycloheptyl group, a cyclooctyl group, a cyclononyl group, a cyclodecyl group, a bicyclo[1.1.0]butyl group, a bicyclo[1.1.1]pentyl group, a bicyclo[2.1.0]pentyl group, a bicyclo[3.1.0]hexyl group, a bicyclo[2.1.1]hexyl group, a bicyclo[2.2.0]hexyl group, a bicyclo[2.2.1]heptyl (norbornyl) group, a bicyclo[3.1.1]heptyl group, a bicyclo[3.2.0]heptyl group, a bicyclo[4.1.0]heptyl group, a bicyclo[2.2.2]octyl group, a bicyclo[3.2.1]octyl group, a bicyclo[3.3.0]octyl group, a bicyclo[4.1.1]octyl group, a bicyclo[4.2.0]octyl group, a bicyclo[5.1.0]octyl group, a bicyclo[3.2.2]nonyl group, a bicyclo[3.3.1]nonyl group, a bicyclo[4.2.1]nonyl group, a bicyclo[4.3.0]nonyl group, a bicyclo[5.1.1]nonyl group, a bicyclo[5.2.0]nonyl group, a bicyclo[6.1.0]nonyl group, a bicyclo[4.3.1]decyl group, a tricyclo[5.2.1.0²,6]decyl group, an isobornyl group, an adamantyl group, or an androstanyl group.

7. The compound of claim 1, wherein, in General Formula 1, Y is a 2-fluorophenyl group, a 3-fluorophenyl group, a 4-fluorophenyl group, a 2,4-difluorophenyl group, a 2,3-difluorophenyl group, a 3,4-difluorophenyl group, a 3,5-difluorophenyl group, a 2,4,5-trifluorophenyl group, a 2,3,4-trifluorophenyl group, a 2,3,4,5-tetrafluorophenyl group, a 2,3,5,6-tetrafluorophenyl group, a 2,3,4,5,6-pentafluorophenyl group, a 2-fluoronaphthyl group, a 3-fluoronaphthyl group, a 4-fluoronaphthyl group, a 2,3,4,5,6,7-hexafluoronaphthyl group, a 2,3,4,5,6,7,8-heptafluoronaphthyl group, a 1,3,4,5,6,7,8-heptafluoronaphthyl group, or a 4,4′-difluorobiphenyl group.

8. The compound of claim 1, wherein, in General Formula 1, Y is —CF₃, —C₄F₉, or a 2,3,4,5,6-pentafluorophenyl group.

9. A polymeric compound comprising a first structural unit derived from a compound represented by General Formula 1: 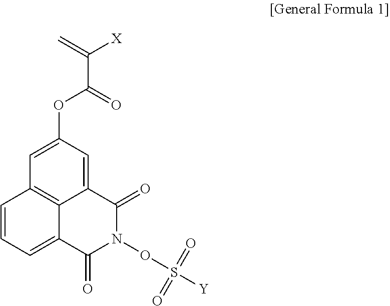
wherein, in General Formula 1,
X is a hydrogen atom or a methyl group, and
Y is: a C1 to C20 linear alkyl group in which at least one hydrogen atom is substituted with a fluorine atom, a C3 to C20 branched alkyl group in which at least one hydrogen atom is substituted with a fluorine atom, an unsubstituted C3 to C20 cycloalkyl group, a C3 to C20 cycloalkyl group in which at least one hydrogen atom is substituted with a fluorine atom, a C3 to C20 cycloalkyl group in which at least one hydrogen atom is substituted with an oxygen atom, an unsubstituted C4 to C21 cycloalkylalkyl group, a C4 to C21 cycloalkylalkyl group in which at least one hydrogen atom is substituted with an oxygen atom, or a C6 to C18 aryl group in which at least one hydrogen atom is substituted with a fluorine atom.

10. The polymeric compound of claim 9, further comprising a second structural unit having an acid-decomposable group.

11. The polymeric compound of claim 10, wherein the second structural unit is represented by Formula (L1-1) or (L1-2): 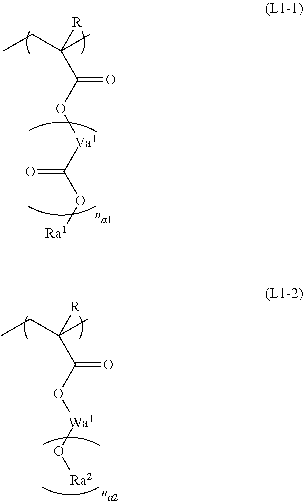
wherein, in each of Formulae (L1-1) and (L1-2), R is a hydrogen atom, a C1 to C5 alkyl group, or a C1 to C5 alkyl halide group;
in Formula (L1-1), Va¹ is a C1 to C10 linear aliphatic hydrocarbon group or a C3 to C10 branched aliphatic hydrocarbon group, nₐ₁ is 0, 1, or 2, and Ra¹ is an acid-labile group; and
in Formula (L1-2), Wa¹ is a 2-valent to 4-valent hydrocarbon group, Ra² is an acid-labile group, and nₐ₂ is 1, 2, or 3.

12. The polymeric compound of claim 10, wherein the second structural unit is represented by Formula (L1-1-1): 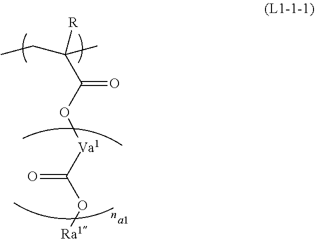
wherein, in Formula (L1-1-1),
R is a hydrogen atom, a C1 to C5 alkyl group, or a C1 to C5 alkyl halide group,
Va¹ is a C1 to C10 linear aliphatic hydrocarbon group or a C3 to C10 branched aliphatic hydrocarbon group,
nₐ₁ is 0, 1, or 2, and
Ra^{1″} is an acid-labile group represented by Formula (L1-r2-1): 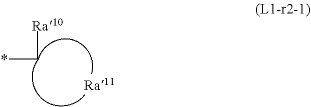
wherein, in Formula (L1-r2-1),
Ra′¹⁰ is a C1 to C12 linear or branched alkyl group, and
Ra′¹¹ is a group forming an aliphatic cyclic group together with a carbon atom bonded with Ra′¹⁰, and Ra′¹¹ is a group obtained by removing one hydrogen atom from cyclopentane, cyclohexane, adamantane, norbornane, isobornane, tricyclodecane, or tetracyclododecane.

13. The polymeric compound of claim 12, wherein at least a portion of Ra′¹⁰ is substituted with a halogen atom or a heteroatom-containing group, the heteroatom being an oxygen atom, a sulfur atom, or a nitrogen atom, and the heteroatom-containing group being —O—, —C(═O)—O—, —O—C(═O)—, —C(═O)—, —O—C(═O)—O—, —C(═O)—NH—, —NH—, —S—, —S(═O)₂—, or —S(═O)₂—O.

14. The polymeric compound of claim 9, further comprising a third structural unit having an ether bond,
wherein the third structural unit is 2-methoxyethyl (meth)acrylate, 2-ethoxyethyl (meth)acrylate, methoxytriethylene glycol (meth)acrylate, 3-methoxybutyl (meth)acrylate, ethyl carbitol (meth)acrylate, phenoxypolyethylene glycol (meth)acrylate, methoxypolyethylene glycol (meth)acrylate, methoxypolypropylene glycol (meth)acrylate, or tetrahydrofurfuryl (meth)acrylate.

15. The polymeric compound of claim 9, further comprising a fourth structural unit including an acid-nonlabile alicyclic group,
wherein the acid-nonlabile alicyclic group of the fourth structural unit includes at least one selected from the group consisting of a tricyclodecyl group, an adamantyl group, a tetracyclododecyl group, an isobornyl group, and a norbornyl group.

16. The polymeric compound of claim 9, wherein, in the polymeric compound, the first structural unit derived from the compound represented by General Formula 1 is present in an amount of 0.1 mol % to 30 mol %, based on a total amount of all structural units of the polymeric compound.

17. The polymeric compound of claim 9, wherein, in General Formula 1, Y is a C1 to C15 fluoroalkyl group or a C1 to C15 partially fluorinated alkyl group.

18. A resist composition comprising:
a polymeric compound that comprises a first structural unit derived from a compound represented by General Formula 1: 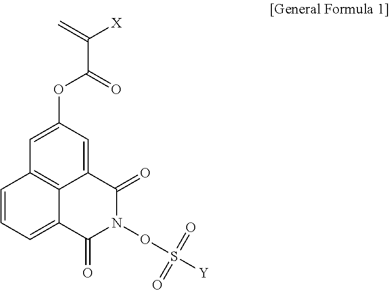
wherein, in General Formula 1,
X is a hydrogen atom or a methyl group, and
Y is: a C1 to C20 linear alkyl group in which at least one hydrogen atom is substituted with a fluorine atom, a C3 to C20 branched alkyl group in which at least one hydrogen atom is substituted with a fluorine atom, an unsubstituted C3 to C20 cycloalkyl group, a C3 to C20 cycloalkyl group in which at least one hydrogen atom is substituted with a fluorine atom, a C3 to C20 cycloalkyl group in which at least one hydrogen atom is substituted with an oxygen atom, an unsubstituted C4 to C21 cycloalkylalkyl group, a C4 to C21 cycloalkylalkyl group in which at least one hydrogen atom is substituted with an oxygen atom, or a C6 to C18 aryl group in which at least one hydrogen atom is substituted with a fluorine atom; and
an organic solvent.

19. The resist composition of claim 18, further comprising an acid generator, an acid diffusion inhibitor, or an adhesion improver.

20. The resist composition of claim 18, further comprising a cross-linking agent.

21.-22. (canceled)
